SEQUENCE LISTING (8 sequences)

SEQ ID NO: 1 - DNA, 45 bp, Artificial
  primer (RegI forward)
gcgcgcaattaaccctcactaaagggagataacagttgtgatgcc

SEQ ID NO: 2 - DNA, 47 bp, Artificial
  primer (RegI reverse)
atggattaatacgactcactatagggtttatttaaatgtgcagggtt

SEQ ID NO: 3 - DNA, 44 bp, Artificial
  primer (RegIII forward)
gcgcgcaattaaccctcactaaagggaaggtcaccgtgacaagg

SEQ ID NO: 4 - DNA, 47 bp, Artificial
  primer (RegIII reverse)
atggattaatacgactcactatagggcaagattgcaaagcaggaact

SEQ ID NO: 5 - DNA, 44 bp, Artificial
  primer (TFF2 forward)
gcgcgcaattaaccctcactaaagggatcttcgaagtgccctgg

SEQ ID NO: 6 - DNA, 47 bp, Artificial
  primer (TFF2 reverse)
atggattaatacgactcactatagggccactgctgaggctcaagaga

SEQ ID NO: 7 - DNA, 40 bp, Artificial
  primer (Ins2 forward)
gcgcgcaattaaccctcactaaagggacccacaagtggca

SEQ ID NO: 8 - DNA, 47 bp, Artificial
  primer (Ins2 reverse)
atggattaatacgactcactatagggttgcagtagttctccagttgg

(19) United States
(12) Patent Application Publication
(10) Pub. No.: US 2004/0176293 A1
(43) Pub. Date: Sep. 9, 2004

(54) Methods for screening, treating and diagnosing inflammatory bowel disease and compositions thereof

(75) Inventors: Ron L. Peterson, North Chelmsford, MA (US); Andrew J. Dorner, Lexington, MA (US)

Correspondence Address: FITZPATRICK CELLA HARPER & SCINTO, 30 ROCKEFELLER PLAZA, NEW YORK, NY, 10112, US

(73) Assignee: Wyeth, Madison, NJ

(21) Appl. No.: 10/740,218

(22) Filed: Dec. 18, 2003

Related U.S. Application Data
(60) Provisional application No. 60/434,338, filed on Dec. 18, 2002.
(60) Provisional application No. 60/434,356, filed on Dec. 18, 2002.
(60) Provisional application No. 60/434,362, filed on Dec. 18, 2002.

(51) Int. Cl. A61K 48/00; A61K 38/17; G01N 33/53; G01N 33/567

(52) U.S. Cl. 514/12; 514/44; 435/7.2

(57) ABSTRACT

Methods and compositions for the identification of novel targets for diagnosis, prognosis, therapeutic intervention and prevention of IBD. In particular, the present invention is directed to the identification of novel targets that are IBD differential markers. The present invention is further directed to methods of high-throughput screening for test compounds capable of modulating the activity of proteins encoded by the novel targets. Moreover, the present invention is also directed to methods that can be used to assess the efficacy of test compounds and therapies for the ability to inhibit IBD. Methods for determining the long-term prognosis in a subject are also provided.

This application claims the benefit of U.S. Provisional Application Ser. Nos. 60/434,338; 60/434,356; and 60/434,362, all filed Dec. 18, 2002, and all incorporated herein by reference in their entireties.

### BACKGROUND OF THE INVENTION

1. Field of the Invention

The present invention is directed to novel methods for diagnosis, prognosis and treatment of inflammatory bowel disease (IBD) using differentially expressed genes. The present invention is further directed to novel therapeutics and therapeutic targets and to methods of screening and assessing test compounds for the treatment and prevention of IBD. In particular, the present invention is directed to methods of modulating the expression levels of genes associated with IBD and modulated by administration of interleukin-11, as well as modulating the activities of proteins corresponding to those genes.

2. Related Background Art

Interleukin-11 (IL-11) is a pleiotropic cytokine shown to have effects on multiple cell and tissue types. It is a member of a family of cytokines that use the membrane glycoprotein gp130 as the signaling receptor subunit, including IL-6, ciliary neurotropic factor, leukemia inhibitory factor, oncostatin M and cardiotropin-1 (Trepicchio et al. (1998) Ann. NY Acad. Sci. 856:12-21; Schwertschlag et al. (1999) Leukemia 13:1307-15). Recombinant human interleukin-11 (rhIL-11) was identified by its activity as a hematopoietic growth factor capable of stimulating multiple stages of megakaryocytopoiesis to increase peripheral platelet levels in normal and myelosuppressed animals (Leonard et al. (1994) Blood 83:1499-1506; Schlerman et al. (1996) Stem Cells 14:517-32; Du et al. (1993) Blood 81:27-34) and is currently approved for the treatment of chemotherapy-induced thrombocytopenia.

In addition to its thrombopoietic activities, rhIL-11 has been shown to be an anti-inflammatory cytokine in multiple in vitro and in vivo models. rhIL-11 inhibits the production of proinflammatory mediators including TNF-α, IL-1β and nitric oxide from activated macrophages through its ability to inhibit the nuclear translocation of the transcription factor NF-κB (Lentsch et al. (1999) Leukoc. Biol. 66:151-57; Trepicchio et al. (1999) J. Clin. Invest. 104:1527-37 (published erratum appears in (2000) J. Clin. Invest. 105:396)). In animal models, rhIL-11 has been shown to downregulate proinflammatory cytokine production. rhIL-11 pretreatment has been shown to reduce the serum levels of TNF-α, IL-1β and IFN-γ in a murine model of endotoxemia (Trepicchio et al. (1996) J. Immunol. 157:3627-34). rhIL-11 also reduced the level of TNF-α mRNA in lung and alveolar macrophages in a murine model of radiation-induced thoracic injury (Redlich et al. (1996) J. Immunol. 157:1705-10). In a rat model of Clostridium difficile enterotoxicity, rhIL-11 treatment also decreased TNF-α production from intestinal macrophages (Castagliuolo et al. (1997) Am. J. Physiol. 273:G333-41). In addition to its effects on macrophages, rhIL-11 promotes T-cell polarization towards the Th2 phenotype in vitro (Bozza et al. (2001) J Interferon Cytokine Res. 21:21-30) and in a murine model of graft-vs.-host disease (Hill et al. (1998) J. Clin. Invest. 102:115-23; Teshima et al. (1999) J. Clin. Invest. 104:317-25).

rhIL-11 also has effects on intestinal epithelial cells. rhIL-11 acts directly on rat intestinal epithelial cells in culture to decrease proliferation through inhibition of pRB phosphorylation (Peterson et al. (1996) Am. J. Pathol. 149:895-902). Animal models have suggested that rhIL-11 acts to maintain the epithelial integrity of the gastrointestinal tract in vivo. In a murine model of severe cytoablative therapy, where mortality was shown to be secondary to sepsis following the destruction of the gastrointestinal epithelial layer, rhIL-11 treatment increased survival (Du et al. (1997) Am. J. Physiol. 272:G545-552). Survival was shown to be associated with the proliferation and decreased apoptosis of intestinal epithelial crypt cells (Orazi et al. (1996) Lab. Invest. 75:33-42). Similarly, in a murine model of massive small bowel resection, rhIL-11 was shown to have a trophic effect on small intestinal enterocytes, causing cell proliferation and increased mucosal thickness (Fiore et al. (1998) J. Pediatr. Surg. 33:24-29). rhIL-11 has also been shown to decrease tissue damage in other acute models of gastrointestinal injury including ischemic bowel necrosis (Du et al. (1997) supra) and trinitrobenzene sulfonic acid (TNB)-induced colitis (Qiu et al. (1996) Dig. Dis. Sci. 41:1625-30).

The HLA-B27 transgenic rat, coexpressing the human major histocompatibility class I allele HLA-B27 and β2-microglobulin, develops T cell-dependent chronic multiorgan inflammatory disease, including inflammatory bowel disease (IBD), reminiscent of human B27-associated spondyloarthropathies (Hammer et al. (1990) Cell 63:1099-1112). rhIL-11 has been shown to ameliorate the IBD in this model (Keith et al. (1994) Stem Cells 12 (suppl. 1):79-90). A previous study of the molecular and cellular effects of rhIL-11 in this model revealed that rhIL-11 treatment reduced the levels of proinflammatory cytokine mRNA in the colon and reduced the levels of myeloperoxidase activity in the intestine (Peterson et al. (1998) Lab. Invest. 78:1503-12). Spleen cells taken from rhIL-11-treated animals produced less TNF-α, IFN-γ and IL-2 upon anti-CD3 antibody activation in vitro than cells derived from control animals, suggestive of rhIL-11 mediated effects on T cells (Peterson et al. (1998) supra). Recently, the HLA-B27 transgenic rat model has been used to produce a pharmacogenomic analysis of rhIL-11 treatment of IBD (Peterson et al. (2002) Pharmacogenomics J 2(6):383-99).

### SUMMARY OF THE INVENTION

The present invention is based on the discovery that the expression of certain genetic markers is altered in tissues from subjects with inflammatory bowel disease (IBD) as compared to normal subjects, as well as the further discovery that a subset of those markers is modulated by treatment of disease with rhIL-11. The present invention provides compounds that modulate the expression of these genetic markers and/or the activities of the proteins encoded by these genetic markers in tissues from subjects with IBD, including, but not limited to, nucleic acid molecules encoding these genetic markers, and homologs, analogs, and deletions thereof, as well as inhibitory polynucleotides, polypeptides, and small molecules. The present invention further provides methods, biochips, and kits for diagnosing, prognosing, and monitoring the course of inflammatory bowel disease based on the aberrant expression of these genetic markers, as well as therapies for use as remedies for such aberrant expression. In addition, the present invention provides pharmaceutical formulations and routes of administration for such remedies, as well as methods for assessing the efficacy of such remedies.

The present invention also is based on the discovery that expression of RegIII and Ins2 is increased in tissues from subjects with inflammatory bowel disease following treatment with rhIL-11. The present invention provides therapeutic compounds that increase the expression and/or activity of RegIII and/or Ins2, alone or in combination with other known or putative epithelial growth factors, in tissues from subjects with inflammatory bowel disease, including, but not limited to, nucleic acid molecules encoding RegIII or Ins2 and homologs, analogs, and deletions thereof, as well as polypeptides and small molecules. The present invention further provides pharmaceutical formulations and routes of administration for such therapeutic compounds, as well as methods for assessing the efficacy of such therapeutic compounds.

The present invention also is based on the discovery that HLA-DMβ/RT1.DMβ expression is increased in tissues from subjects with inflammatory bowel disease as compared to normal subjects, as well as the further discovery that such increased expression of HLA-DMβ/RT1.DMβ is decreased by treatment of disease with rhIL-11. The present invention provides compounds that inhibit the expression and/or activity of HLA-DMβ or RT1.DMβ in tissues from subjects with inflammatory bowel disease, including, but not limited to, nucleic acid molecules encoding HLA-DMβ or RT1.DMβ and homologs, analogs, and deletions thereof, as well as inhibitory polynucleotides, polypeptides, and small molecules. The present invention further provides methods and kits for diagnosing, prognosing, and monitoring the course of inflammatory bowel disease based on the aberrant gene expression of HLA-DMβ or RT1.DMβ, as well as therapies for use as remedies for such aberrant expression. In addition, the present invention provides pharmaceutical formulations and routes of administration for such remedies, as well as methods for assessing the efficacy of such remedies.

### BRIEF DESCRIPTION OF THE DRAWINGS

FIG. 1: Effect of rhIL-11 on Stool Character Analysis

Stool character of vehicle- and rhIL-11-treated HLA-B27 rats was observed daily and scored as normal (cross-hatched boxes), soft (shaded boxes), or diarrhea (unfilled boxes). Groups of vehicle- and rhIL-11-treated rats were killed after 3 and 4 days of treatment for analysis. rhIL-11-treated rats exhibited much fewer days of diarrhea compared to vehicle-treated rats. Normal stool character was observed only in the rhIL-11-treated rats beginning on the second day of treatment and persisted in most of the data through the end of the study. Asterisks (*) denote the days of subcutaneous administration of rhIL-11 or vehicle; a pound sign (#) denotes the day of BrdU injection.

FIG. 2: Increased BrdU Incorporation in Colonic Epithelial Cells of rhIL-11-Treated Rats

BrdU-containing cells were identified by immunohistochemistry with anti-BrdU antibody. Sections of colonic tissue were analyzed for the presence and quantification of BrdU positive cells by counting five crypts per slide (i.e., five crypts per animal) and calculating the percentage of BrdU positive cells/total number of epithelial cells. Data was analyzed by one-way analysis of variance (ANOVA) and Tukey's multiple comparison test, using GraphPad Prism™ software; and significance was measured at p<0.01 (***). Significantly more cells were labeled with BrdU in the colons of HLA-B27 rats treated with rhIL-11 than rats treated with vehicle, indicating that rhIL-11 caused increased proliferation of intestinal epithelial cells.

### DETAILED DESCRIPTION OF THE INVENTION

The present invention provides for the identification of novel targets and therapeutics for the intervention and prevention of inflammatory bowel disease (IBD). In particular, the present invention provides methods for the identification of novel therapeutic targets to be analyzed in high-throughput screening assays of test compounds capable of preventing or treating IBD. The present invention further provides methods and compositions for the identification of novel targets for diagnosis, prognosis, therapeutic intervention and prevention of IBD. In particular, the present invention provides the identification of novel targets that are IBD differential markers. Moreover, the present invention provides methods that can be used to assess the efficacy of test compounds and therapies for the ability to inhibit IBD. Methods for determining the long-term prognosis in a subject having IBD are also provided. The present invention also includes the use of RegIII and Ins2, as well as other epithelial growth factors and putative growth factors, as therapeutic agents. The present invention also provides for the inhibition of HLA-DMβ/RT1.DMβ as therapeutic treatment of IBD. In addition, the invention provides disease-related genes that can be useful for diagnosis of IBD, as well as drug-responsive genes that can be useful as indicators of healing.

In one embodiment, the invention provides a method of diagnosing a subject with IBD, the method comprising the step of comparing a level of expression of at least one IBD marker in a sample from the subject, wherein the IBD marker is listed in Table 2; and a normal level of expression of the at least one IBD marker in a control sample, wherein a substantial difference between the level of expression of the IBD marker(s) in the sample from the subject and the normal level is an indication that the subject is afflicted with IBD. In a preferred embodiment, the sample is collected from the group consisting of duodenum, ileum, jejunum, colon and rectum. In another preferred embodiment, the sample is collected from feces. In another preferred embodiment, the control sample is from a nondiseased subject and the substantial difference is a factor of at least about 2-fold. In another preferred embodiment, the control sample is from nondiseased tissue of the subject and the substantial difference is a factor of at least about 2-fold. In another preferred embodiment, the level of expression of the at least one IBD marker in the sample is assessed by detecting the presence in the sample of a protein or portion thereof corresponding to the IBD marker(s). In another preferred embodiment, the level of expression of the at least one IBD marker in the sample is assessed by detecting the presence in the sample of a transcribed polynucleotide or portion thereof, wherein the transcribed polynucleotide comprises the IBD marker(s). In another preferred embodiment, the level of expression of the at least one IBD marker in the sample is assessed by detecting the presence in the sample of a transcribed polynucleotide or a portion thereof that hybridizes to a labeled probe under highly stringent conditions, wherein the transcribed polynucleotide comprises the IBD marker(s). In particularly preferred embodiment, the at least one IBD marker is selected from the group consisting of RT1.DMβ and HLA-DMβ (the latter being the human ortholog of the former). In another preferred embodiment, the at least one IBD marker is a plurality of IBD markers. In a further preferred embodiment, the plurality of IBD markers comprises at least five IBD markers.

In another embodiment, the invention provides a method of assessing the efficacy of a test compound for inhibiting IBD in a subject comprising the step of comparing a level of expression of an IBD marker, wherein the IBD marker is listed in Tables 4 or 5, in a first sample obtained from the subject, wherein the first sample is exposed to the test compound; and a level of expression of the same IBD marker in a second sample obtained from the subject, wherein the second sample is not exposed to the test compound, wherein a substantially modulated level of expression of the IBD marker in the first sample, relative to the second sample, is an indication that the test compound is efficacious for inhibiting IBD in the subject. In a preferred embodiment, the IBD marker is selected from the group consisting of HLA-DMβ and RT1.DMβ, and the substantially modulated level of expression is a substantially decreased level of expression. In another preferred embodiment, the IBD marker is selected from the group consisting of RegIII and Ins2, and the substantially modulated level of expression is a substantially increased level of expression. In another embodiment, the invention provides a method of identifying a test compound for inhibiting IBD comprising the step of comparing a level of expression of an IBD marker, wherein the IBD marker is listed in Tables 4 or 5, in a first sample, wherein the first sample is contacted with one of a plurality of test compound; and a level of expression of the same IBD marker in a second sample, wherein the second sample is not contacted with the test compound, wherein a substantially modulated level of expression of the IBD marker in the first sample, relative to the second sample, is an indication that the test compound is efficacious for inhibiting IBD. In a preferred embodiment, the IBD marker is selected from the group consisting of HLA-DMβ and RT1.DMβ, and the substantially modulated level of expression is a substantially decreased level of expression. In another preferred embodiment, the IBD marker is selected from the group consisting of RegIII and Ins2, and the substantially modulated level of expression is a substantially increased level of expression.

In another embodiment, the invention provides a method of assessing the efficacy of a therapy for inhibiting IBD in a subject comprising the step of comparing a level of expression of an IBD marker, wherein the IBD marker is listed in Tables 4 or 5, in a first sample obtained from the subject prior to providing at least a portion of the therapy to the subject; and a level of expression of the same IBD marker in a second sample following provision of the portion of the therapy, wherein a substantially modulated level of expression of the IBD marker in the second sample, relative to the first sample, is an indication that the therapy is efficacious for inhibiting IBD in the subject. In a preferred embodiment, the IBD marker is selected from the group consisting of HLA-DMβ and RT1.DMβ, and the substantially modulated level of expression is a substantially decreased level of expression. In another preferred embodiment, the IBD marker is selected from the group consisting of RegIII and Ins2, and the substantially modulated level of expression is a substantially increased level of expression.

In another embodiment, the invention provides a method of screening for test compounds capable of modulating the expression of an IBD marker gene product encoded by an IBD marker listed in Table 2, the method comprising contacting a sample containing the IBD marker gene product with a plurality of test compounds; and determining whether expression of the IBD marker gene product in the sample is modulated relative to the expression of the IBD marker gene product in a sample not contacted with the test compound, wherein a modulation of expression indicates that the test compound inhibits IBD. In a preferred embodiment, the IBD marker is selected from the group consisting of HLA-DMβ and RT1.DMβ.

In another embodiment, the invention provides a method of screening for test compounds capable of inhibiting IBD, the method comprising combining an IBD marker protein encoded by an IBD marker listed in Tables 4 or 5, a binding partner of the IBD marker protein, and a test compound; selecting one of the test compounds that modulates the binding of the IBD marker protein and the binding partner of the IBD marker protein as compared to other test compounds; and correlating the amount of modulation of binding with the ability of the test compound to inhibit IBD, wherein modulation of binding of the IBD marker protein and the binding partner of the IBD marker protein indicates that the test compound is capable of inhibiting IBD. In a preferred embodiment, the IBD marker is selected from the group consisting of HLA-DMβ and RT1.DMβ. In another preferred embodiment, the step of selecting comprises detecting binding of one of the test compounds to the IBD marker protein. In another preferred embodiment, the step of selecting comprises detecting binding of one of the test compounds to the binding partner of the IBD marker protein.

In another embodiment, the invention provides a method of screening test compounds for inhibitors of IBD in a subject, the method comprising the steps of obtaining a sample comprising cells; contacting an aliquot of the sample with one of a plurality of test compounds; comparing a level of expression of an IBD marker listed in Tables 2, 4 or 5; and selecting one of the test compounds that substantially modulates the level of expression of the IBD marker in the aliquot containing that test compound, relative to other test compounds. In a preferred embodiment, the IBD marker is selected from the group consisting of HLA-DMβ and RT1.DMβ.

In another embodiment, the invention provides a method of treating a subject diagnosed with IBD, the method comprising administering a composition comprising a compound that modulates the activity of an IBD marker polypeptide and a pharmaceutically acceptable carrier, wherein the IBD marker is listed in Table 4 and the expression of the IBD marker was modulated by rhIL-11 treatment in Table 4. In a preferred embodiment, the IBD marker is selected from the group consisting of HLA-DMβ and RT1.DMβ.

In another embodiment, the invention provides a method of treating a subject diagnosed with IBD, the method comprising administering a composition comprising an IBD marker polypeptide and a pharmaceutically acceptable carrier, wherein the IBD marker is listed in Table 4 and the expression of the IBD marker was increased by rhIL-11 treatment in Table 4.

In another embodiment, the invention provides a method of treating a subject diagnosed with IBD, the method comprising administering to the subject an isolated nucleic acid molecule encoding an IBD marker listed in Table 4 operably linked to at least one expression control sequence, wherein the IBD marker protein is expressed in the subject, and wherein the IBD marker is listed in Table 4 and the expression of the IBD marker was increased by rhIL-11 treatment in Table 4.

In another embodiment, the invention provides a method of decreasing a level of expression of an IBD marker, the method comprising providing to cells of a subject a polynucleotide that inhibits expression of an IBD marker, wherein the IBD marker is listed in Table 2 and the expression of the IBD marker was increased in the diseased rat colon in Table 2. In a preferred embodiment, the IBD marker is selected from the group consisting of HLA-DMβ and RT1.DMβ.

In another embodiment, the invention provides a method of decreasing a level of expression of an IBD marker, the method comprising providing to cells of a subject a siRNA molecule that inhibits expression of an IBD marker, wherein the siRNA molecule is targeted to a mRNA corresponding to an IBD marker listed in Table 2 and the expression of the IBD marker was increased in the diseased rat colon in Table 2. In a preferred embodiment, the IBD marker is selected from the group consisting of HLA-DMβ and RT1.DMβ.

In another embodiment, the invention provides a method of decreasing a level of expression of an IBD marker, the method comprising providing to cells of a subject an antisense oligonucleotide complementary to an IBD marker, wherein the IBD marker is listed in Table 2 and the expression of the IBD marker was increased in the diseased rat colon in Table 2. In a preferred embodiment, the IBD marker is selected from the group consisting of HLA-DMβ and RT1.DMβ.

In another embodiment, the invention provides a method of decreasing activity of an IBD marker protein encoded by an IBD marker, the method comprising providing to cells of a subject an antibody capable of immunospecific binding to an IBD marker protein, wherein the IBD marker protein is encoded by an IBD marker listed in Table 2. In a preferred embodiment, the IBD marker is selected from the group consisting of HLA-DMβ and RT1.DMβ.

In another embodiment, the invention provides a method of localizing a therapeutic moiety to tissue having IBD, the method comprising linking the therapeutic moiety to a binding partner of an IBD marker protein encoded by an IBD marker listed in Tables 4 or 5, wherein the binding partner is selected from the group consisting of an antibody that is capable of immunospecific binding to the IBD marker protein and an IBD protein ligand; and administering to a subject in need of treatment the therapeutic moiety linked to the binding partner. In a preferred embodiment, the IBD marker is selected from the group consisting of HLA-DMβ and RT1.DMβ.

In another embodiment, the invention provides a method of localizing a therapeutic moiety to tissue having IBD, the method comprising linking a therapeutic agent to a binding partner of an IBD marker, wherein the marker is listed in Table 2; and administering to a subject in need of treatment the therapeutic moiety linked to the binding partner. In a preferred embodiment, the IBD marker is selected from the group consisting of HLA-DMβ and RT1.DMβ.

In another embodiment, the invention provides a biochip comprising at least five or more IBD markers listed in Tables 4 or 5, wherein the biochip is utilized in high-throughput screening assays for inhibition of IBD. In another embodiment, the invention provides a biochip comprising at least five or more IBD markers listed in Table 2, wherein the biochip is utilized in diagnosing IBD.

In another embodiment, the invention provides a composition capable of inhibiting IBD in a subject, the composition comprising an IBD marker polypeptide and a pharmaceutically acceptable carrier, wherein the IBD marker polypeptide is encoded by an IBD marker listed in Table 4 and the expression of the IBD marker was increased by rhIL-11 treatment in Table 4.

In another embodiment, the invention provides a composition capable of inhibiting IBD in a subject, the composition comprising a siRNA molecule and a pharmaceutically acceptable carrier, wherein the siRNA molecule is targeted to a mRNA corresponding to an IBD marker listed in Table 4 and the expression of the IBD marker was decreased by rhIL-11 treatment in Table 4. In a preferred embodiment, the IBD marker is selected from the group consisting of HLA-DMβ and RT1.DMβ.

In another embodiment, the invention provides a kit for determining the long-term prognosis in a subject having IBD, the kit comprising a polynucleotide probe, wherein the probe specifically binds to a transcribed IBD marker polynucleotide, wherein the IBD marker is listed in Tables 2, 4 or 5. In a preferred embodiment, the IBD marker is selected from the group consisting of HLA-DMβ and RT1.DMβ.

In another embodiment, the invention provides a kit for determining the long-term prognosis in a subject having IBD, the kit comprising an antibody capable of immunospecific binding to a protein encoded by an IBD marker listed in Tables 2, 4 or 5. In a preferred embodiment, the IBD marker is selected from the group consisting of HLA-DMβ and RT1.DMβ.

In another embodiment, the invention provides a kit comprising a biochip and a computer readable medium, wherein the biochip comprises at least two IBD markers listed in Tables 2, 4 or 5 and wherein the computer readable medium contains the same IBD markers in computer readable form.

In another embodiment, the invention provides a kit for diagnosing IBD in a subject, the kit comprising a polynucleotide probe wherein the probe specifically binds to a transcribed IBD marker polynucleotide, wherein the IBD marker is listed in Table 2. In a preferred embodiment, the IBD marker is selected from the group consisting of HLA-DMβ and RT1.DMβ.

In another embodiment, the invention provides a kit for diagnosing IBD in a subject, the kit comprising an antibody capable of immunospecific binding to a protein encoded by an IBD marker listed in Table 2. In a preferred embodiment, the IBD marker is selected from the group consisting of HLA-DMβ and RT1.DMβ.

In another embodiment, the invention provides a method of monitoring the progression of IBD in a subject, the method comprising the steps of detecting in a subject sample, at a first point in time, a level of expression of at least one IBD marker, wherein the IBD marker is listed in Table 2; detecting in a subject sample, at a second point in time, a level of expression of the same IBD marker(s); and detecting a substantial difference between the levels of expression of the IBD marker(s) between the first point in time and the second point in time, wherein the substantial difference between the levels of expression indicates that the subject has progressed to a different stage of IBD. In a preferred embodiment, the at least one IBD marker is selected from the group consisting of HLA-DMβ and RT1.DMβ.

In another embodiment, the invention provides a method of treating a subject diagnosed with IBD, the method comprising providing to the subject a polynucleotide that inhibits expression of an IBD marker, wherein the IBD marker is listed in Table 4 and the expression of the IBD marker was decreased by rhIL-11 treatment in Table 4. In a preferred embodiment, the IBD marker is selected from the group consisting of HLA-DMβ and RT1.DMβ.

In another embodiment, the invention provides a method of treating a subject diagnosed with IBD, the method comprising providing to the subject a siRNA molecule that inhibits expression of an IBD marker, wherein the siRNA molecule is targeted to a mRNA corresponding to an IBD marker listed in Table 4 and the expression of the IBD marker was decreased by rhIL-11 treatment in Table 4. In a preferred embodiment, the IBD marker is selected from the group consisting of HLA-DMβ and RT1.DMβ.

In another embodiment, the invention provides a method of treating a subject diagnosed with IBD, the method comprising providing to the subject an antisense oligonucleotide complementary to an IBD marker, wherein the IBD marker is listed in Table 4 and the expression of the IBD marker was decreased by rhIL-11 treatment in Table 4. In a preferred embodiment, the IBD marker is selected from the group consisting of HLA-DMβ and RT1.DMβ.

In another embodiment, the invention provides a method of treating a subject diagnosed with IBD, the method comprising providing to the subject an antibody capable of immunospecific binding to an IBD marker protein, wherein the IBD marker protein is encoded by an IBD marker listed in Table 4 and the expression of the IBD marker was decreased by rhIL-11 treatment in Table 4. In a preferred embodiment, the IBD marker is selected from the group consisting of HLA-DMβ and RT1.DMβ.

In another embodiment, the invention provides a method for determining whether a subject can be effectively treated with a compound for treating IBD, the method comprising the step of comparing a level of expression of at least one IBD marker in a sample from the subject, wherein the IBD marker(s) is listed in Table 2; and a level of expression of the same IBD marker(s) in a sample from another subject known to respond favorably to the compound for treatment of IBD, wherein a similar level of expression of the IBD marker(s) in the two samples is an indication that the subject can be effectively treated for IBD with the compound. In a preferred embodiment, the at least one IBD marker is selected from the group consisting of HLA-DMβ and RT1.DMβ.

In another embodiment, the invention provides a method of treating a subject suffering from IBD comprising administering RegIII protein or Ins2 protein to the subject.

In another embodiment, the invention provides a method of treating a subject suffering from IBD comprising administering to the subject a plurality of proteins selected from the group consisting of RegIII, Ins2, RegI, and TFF-2, provided said plurality of proteins does not contain only RegI and TFF-2. In a preferred embodiment, the plurality of proteins comprises a combination of proteins.

In another embodiment, the invention provides a method of treating a subject diagnosed with IBD, the method comprising administering to the subject an isolated nucleic acid molecule encoding RegIII or Ins2 operably linked to at least one expression control sequence, wherein the RegIII protein or the Ins2 protein is expressed in the subject.

In another embodiment, the invention provides a method of treating a subject diagnosed with IBD comprising administering to the subject a plurality of isolated nucleic acid molecules encoding proteins selected from the group consisting of RegIII, Ins2, RegI, and TFF-2, operably linked to at least one expression control sequence, provided said plurality of isolated nucleic acid molecules encoding proteins does not contain only RegI and TFF-2. In a preferred embodiment, the plurality of isolated nucleic acid molecules encoding proteins comprises a combination of isolated nucleic acid molecules encoding proteins.

In another embodiment, the invention provides a method of identifying a compound capable of increasing the activity of a protein selected from the group consisting of RegIII and Ins2 comprising the steps of contacting a sample containing the protein with one of a plurality of test compounds; and comparing the activity of the protein in the contacted sample with that in a sample containing the protein not contacted with the test compound, wherein a substantial increase in the activity of the protein in the contacted sample identifies the compound as an activator of protein activity useful in treating IBD. In another preferred embodiment, the invention provides a method of treating a subject suffering from IBD comprising administering to the subject a compound identified by the provided method. In another embodiment, the invention provides a method of identifying a compound capable of increasing the expression of an IBD marker selected from the group consisting of RegIII and Ins2 comprising the steps of contacting a sample containing the IBD marker with one of a plurality of test compounds; and comparing the level of expression of the IBD marker in the contacted sample with that in a sample containing the IBD marker not contacted with the test compound, wherein a substantial increase in the level of expression of the IBD marker in the contacted sample identifies the compound as useful in treating IBD. In another embodiment, the invention provides a method of treating a subject diagnosed with IBD comprising administering to the subject a compound identified by the provided method.

In another embodiment, the invention provides a method of identifying a compound capable of increasing the activities of a plurality of proteins selected from the group consisting of RegIII, Ins2, RegI, and TFF-2, provided said plurality of proteins does not contain only RegI and TFF-2, comprising the steps of contacting a sample containing the plurality of proteins with one of a plurality of test compounds; and comparing the activities of the plurality of proteins in the contacted sample with those in a sample containing the plurality of proteins not contacted with the test compound, wherein increases in the activities of the plurality of proteins in the contacted sample identify the compound as an activator of protein activity useful in treating IBD. In a preferred embodiment, the plurality of proteins comprises a combination of proteins. In another preferred embodiment, the invention provides a method of treating a subject suffering from IBD comprising administering to the subject a compound identified by the provided methods.

In another embodiment, the invention provides a method of treating a subject suffering from IBD comprising administering to the subject a compound that increases the activity of RegIII protein and/or the activity of Ins2 protein.

IBD Differential Markers

In one aspect, the present invention is based on the identification of a number of genetic markers that are differentially expressed in tissue samples from HLA-B27 rats, relative to tissue samples from control nondiseased Fischer 344 rats. These markers may in turn be components of the IBD pathway and thus may serve as diagnostic markers and novel therapeutic targets for treatment of IBD. The expression levels of genes that were differentially expressed between tissues from HLA-B27 rats and Fischer 344 rats at different time points, as well as genes modulated in response to treatment with rhIL-11, are set forth in Tables 2, 4 and 5. These genes and their corresponding gene products (and detectable fragments thereof) are hereinafter known as “IBD markers” or “IBD differential markers.”

In general, Table 2 provides IBD differential markers that are expressed at abnormally increased or decreased levels in tissues from HLA-B27 rats compared to tissues from control Fischer 344 rats, and represent IBD-related genes. In general, Table 4 provides IBD markers from the HLA-B27 rat that are modulated as a result of efficacious treatment with rhIL-11 and may particularly be components of the disease pathway and consequently novel therapeutic targets for treatment and prevention of IBD. The IBD markers listed in Table 4 (except RT1.DMβ) can be viewed as indicators of healing. The markers listed in Tables 2 or 4, which are differentially expressed in HLA-B27 rats, have not been previously associated with IBD. The markers listed in Table 5 previously have not been shown to be differentially expressed upon treatment with rhIL-11.

It is specifically intended by the invention and understood that the IBD markers of the invention also specifically encompass human homologs (or orthologs) of the IBD markers listed in Tables 2 and 5. Markers from other organisms may also be useful in experiments involving animal models for the study of IBD and for drug evaluation. Markers from other organisms may be obtained using the techniques outlined below.

The genes that are known in the art to be linked to IBD may also serve as validation in expression studies for IBD in conjunction with the IBD markers of the invention. The markers that were known prior to the invention to be associated with IBD are provided in Table A. These markers are not to be considered as IBD markers of the invention. However, these markers may be conveniently used in combination with the markers of the invention (e.g., IBD markers listed in Table 2) in the methods, panels, kits and compositions of the invention.

TABLE A

| Accession | Symbol | Fold Δ | P value |
|---|---|---|---|
| Z49761 | RT1-DMα | 5.5637 | 2.6E−05 |
| X14254 | Cd74 | 4.2576 | 8.9E−06 |
| X53054 | RT1-Db1 | 3.028 | 0.00682 |
| U75412 | Igh-4 | 3.8095 | 1.7E−05 |
| M87786 | M87786 | 2.648 | 0.00311 |
| U22424 | Hsd11b2 | −3.415 | 2E−05 |
| S55427 | Pmp22 | −6.4 | 0.0051 |
| K03243 | Pck1 | −21.15 | 0.0076 |
| M18854 | M18854 | 2.7519 | 0.0005 |
| J00771 | Rib1 | −50.65 | 0.0504 |
| X51529 | Pla2g2a | 4.7714 | 2.2E−06 |
| X91234 | Hsd17b2 | −10.65 | 0.00791 |
| S56936 | Ugt1 | −7.5 | 0.00696 |
| L18948 | S100a9 | 5 | 0.003 |
| X70369 | Co13a1 | −2.835 | 0.0471 |
| X76489 | Cd9 | −4.492 | 0.0107 |
| M36151 | RT1-Bb | 4.9675 | 1.93E−05 |
| X07551 | RT1-Ba | 4.7585 | 4.5E−05 |
| U16025 | RT1-M3 | 2.7869 | 2.36E−05 |
| M15562 | RT1-Da | 2.5427 | 0.000184 |
| M98049 | Pap1 | 38.225 | 3.46E−07 |
| L20869 | Pap3 | 17.856 | 5.92E−06 |

Isolated Polynucleotides

The present invention provides isolated polynucleotides and polypeptides as IBD markers, i.e., the invention provides isolated polynucleotides encoding proteins associated with IBD. Preferred nucleotide sequences of the invention include genomic, cDNA, mRNA, siRNA, and chemically synthesized nucleotide sequences.

The IBD markers of the invention are listed in Tables 2 and 5. The invention encompasses polynucleotides sequences of the IBD markers listed in Tables 2 or 5. Polynucleotides of the present invention also include polynucleotides that hybridize under stringent conditions to the polynucleotides sequences of the markers listed in Tables 2 or 5, or their complements, and/or encode polypeptides that retain substantial biological activity (i.e., active fragments) of the markers listed in Tables 2 or 5. Polynucleotides of the present invention also include continuous portions of the polynucleotide sequences of the IBD markers listed in Tables 2 or 5 comprising at least 21 consecutive nucleotides.

The invention further encompasses the polypeptides of the IBD markers listed in Tables 2 or 5. Polypeptides of the present invention also include continuous portions of the polypeptides of the IBD markers set forth in Tables 2 or 5 comprising at least 7 consecutive amino acids. A preferred embodiment of the invention includes any continuous portion of any of the polypeptides of the IBD markers set forth in Tables 2 or 5 that retains substantial biological activity of any of the IBD markers listed in Tables 2 or 5.

The invention further encompasses polynucleotide molecules that differ from the polynucleotide sequences of the IBD markers listed in Tables 2 or 5 only due to the well-known degeneracy of the genetic code, and which thus encode the same proteins as those encoded by the IBD markers listed in Tables 2 or 5.

The polynucleotides encompassed by the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridization, in situ hybridization, and Northern hybridization, and are well known to those skilled in the art.

Hybridization reactions can be performed under conditions of different stringency. The stringency of a hybridization reaction includes the difficulty with which any two nucleic acid molecules will hybridize to one another. The present invention also includes polynucleotides capable of hybridizing under reduced stringency conditions, more preferably stringent conditions, and most preferably highly stringent conditions, to polynucleotides described herein. Examples of stringency conditions are shown in Table B below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE B
Stringency Conditions

| | | Hybrid | | Wash |
|---|---|---|---|---|
| Stringency | Polynucleotide | Length | Hybridization Temperature and | Temperature and |
| Condition | Hybrid | (bp)¹ | Buffer² | Buffer² |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1 xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of | | | | |
| # G + C bases). For hybrids between 18 and 49 base pairs in length, Tₘ(° C.) = 81.5 + 16.6(log₁₀Na⁺) + 0.41 (% G + C) − (600/N), where N is the number of bases in the hybrid, and Na⁺ is the concentration of sodium ions in the hybridization buffer (Na⁺ for 1xSSC = 0.165M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

The polynucleotides of the present invention may also be used as hybridization probes and primers to identify and isolate DNAs having sequences encoding polypeptides homologous to the disclosed polynucleotides. These homologs are polynucleotides and polypeptides isolated from different species than that of the disclosed polynucleotides and polypeptides, or within the same species, but with significant sequence similarity to the disclosed polynucleotides and polypeptides. Preferably, polynucleotide homologs have at least 60% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species, most preferably those isolated from humans.

The polynucleotides of the present invention may be used as hybridization probes and primers to identify and isolate DNAs having sequences encoding allelic variants of the polynucleotides sequences of the IBD markers listed in Tables 2 or 5. Allelic variants are naturally occurring alternative forms of the polynucleotide sequences of the IBD markers listed in Tables 2 or 5 that encode polypeptides that are identical to or have significant similarity to the polypeptides encoded by the genes listed in Tables 2 or 5. Preferably, allelic variants have at least 90% sequence identity (more preferably, at least 95% identity; most preferably, at least 99% identity) with the disclosed polynucleotides.

Consequently, in addition to polynucleotide sequences listed in Tables 2 or 5, the present invention also encompasses homologs and allelic variants of the IBD markers listed in Tables 2 or 5.

The polynucleotides of the present invention may also be used as hybridization probes and primers to identify cells and tissues that express the polypeptides of IBD markers of the present invention and the conditions under which they are expressed.

Additionally, the polynucleotides of the present invention may be used to alter (i.e., enhance, reduce or modify) the expression of the genes corresponding to the IBD markers of the present invention in a cell or organism. These corresponding genes are the genomic DNA sequences of the present invention that are transcribed to produce the mRNAs from which the IBD differential marker polypeptides of the present invention are derived.

Altered expression of the genes encompassed by the present invention in a cell or organism may be achieved through the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med 1:575-88; Knauert and Glazer (2001) Hum. Mol. Genet. 10:2243-51; Bass (2001) Nature 411:428-29).

The inhibitory antisense or ribozyme polynucleotides of the invention can be complementary to an entire coding strand of a gene of the invention, or to only a portion thereof. Alternatively, inhibitory polynucleotides can be complementary to a noncoding region of the coding strand of a gene of the invention. The inhibitory polynucleotides of the invention can be constructed using chemical synthesis and enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages. (Galderisi et al., supra; Heasman (2002) Dev. Biol. 243:209-14; Mickelfield (2001) Curr. Med. Chem. 8:1157-79). Alternatively, antisense molecules can be produced biologically using an expression vector into which a polynucleotide of the present invention has been subcloned in an antisense (i.e., reverse) orientation.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) encompassed by the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In a preferred embodiment, the inhibitory polynucleotide of the present invention is a short interfering RNA (siRNA). siRNAs are short (preferably 20-25 nucleotides; most preferably 21 nucleotides), double-stranded RNA molecules that cause sequence-specific degradation of target mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNAs targeted to Fas mRNA (Song et al. (2003) Nature Med. 9:347-51).

The siRNA molecules of the present invention can be generated by annealing two complementary single-stranded RNA molecules together (one of which matches a portion of the target mRNA) (e.g., Fire et al., U.S. Pat. No. 6,506,559) or through the use of a single hairpin RNA molecule which folds back on itself to produce the requisite double-stranded portion (e.g., Yu et al. (2002) Proc. Natl. Acad. Sci. USA 99:6047-52). The siRNA molecules can be chemically synthesized (Elbashir et al. (2001) Nature 411:494-98) or produced by in vitro transcription using single-stranded DNA templates (e.g., Yu et al. (2002) supra). Alternatively, the siRNA molecules can be produced biologically, either transiently (e.g., Yu et al. (2002) supra; Sui et al. (2002) Proc. Natl. Acad. Sci. USA 99:5515-20) or stably (e.g., Paddison et al. (2002) Proc. Natl. Acad. Sci. USA 99:1443-48), using an expression vector(s) containing the sense and antisense siRNA sequences.

The siRNA molecules targeted to the polynucleotides of the present invention can be designed based on criteria well known in the art (e.g., Elbashir et al. (2001) EMBO J. 20:6877-88). For example, the target segment of the target mRNA should begin with AA (preferred), TA, GA, or CA; the GC ratio of the siRNA molecule should be 45-55%; the siRNA molecule should not contain three of the same nucleotides in a row; the siRNA molecule should not contain seven mixed G/Cs in a row; and the target segment should be in the ORF region of the target mRNA and should be at least 75 bp after the initiation ATG and at least 75 bp before the stop codon. siRNA molecules targeted to the polynucleotides of the present invention can be designed by one of ordinary skill in the art using the aforementioned criteria or other known criteria.

Altered expression of the genes of IBD markers of the present invention in a cell or organism may also be achieved through the creation of nonhuman transgenic animals into whose genomes polynucleotides of the present invention have been introduced. Such transgenic animals include animals that have multiple copies of a gene (i.e., the transgene) of the present invention. A tissue-specific regulatory sequence(s) may be operably linked to the transgene to direct expression of a polypeptide of the present invention to particular cells or a particular developmental stage. In another embodiment, transgenic nonhuman animals can be produced that contain selected systems that allow for regulated expression of the transgene. One example of such a system known in the art is the cre/loxP recombinase system of bacteriophage P1. Methods for generating transgenic animal via embryo manipulation and microinjection, particularly animals such as mice, have become conventional and are well known in the art (e.g., Bockamp et al. (2002) Physiol. Genomics 11:115-32).

Altered expression of the genes of the present invention in a cell or organism may also be achieved through the creation of animals whose endogenous genes corresponding to the polynucleotides of the present invention have been disrupted through insertion of extraneous polynucleotides sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40). In preferred embodiments of the invention, the nonhuman transgenic animals comprise an IBD differential marker. In another preferred embodiment, the nonhuman knockout animal is a RT1.DMβ (or homolog thereof) knockout.

Isolated Polypeptides

Several aspects of the invention pertain to isolated IBD differential marker proteins, and biologically active portions thereof, as well as polypeptide fragments suitable for use as immunogens to raise anti-marker protein antibodies. In one embodiment, native marker proteins can be isolated from cells or tissue sources by an appropriate purification scheme using standard protein purification techniques. In another embodiment, marker proteins are produced by recombinant DNA techniques. Alternative to recombinant expression, a marker protein or polypeptide can be synthesized chemically using standard peptide synthesis techniques.

The IBD marker proteins listed in Tables 2 or 5 may be recombinantly produced by operably linking the polynucleotide sequences of IBD the markers listed in Tables 2 or 5 to an expression control sequence (e.g., the pMT2 and pED expression vectors). General methods of expressing recombinant proteins are well known in the art.

A number of cell lines may act as suitable host cells for recombinant expression of IBD marker polypeptides of the present invention. Mammalian host cell lines include, for example, COS cells, CHO cells, 293T cells, A431 cells, 3T3 cells, CV-1 cells, HeLa cells, L cells, BHK21 cells, HL-60 cells, U937 cells, HaK cells, Jurkat cells, normal diploid cells, as well as cell strains derived from in vitro culture of primary tissue and primary explants.

Alternatively, it may be possible to recombinantly produce the polypeptides of the present invention in lower eukaryotes, such as yeast, or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium. If the polypeptides of the present invention are made in yeast or bacteria, it may be necessary to modify them by, for example, phosphorylation or glycosylation of appropriate sites in order to obtain functionality. Such covalent attachments may be accomplished using well-known chemical or enzymatic methods.

In another embodiment of the invention, IBD marker polypeptides of the present invention may also be recombinantly produced by operably linking the IBD marker polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MaxBac® kit, Invitrogen, Carlsbad, Calif.).

Following recombinant expression in the appropriate host cell, the polypeptides of the present invention may then be purified from culture medium or cell extracts using well-known purification processes, such as gel filtration and ion exchange chromatography. Purification may also include affinity chromatography with agents known to bind the polypeptides of the present invention. These purification processes may also be used to purify the polypeptides of the present invention from natural sources.

Alternatively, the polypeptides of the present invention may also be expressed recombinantly in a form that facilitates identification, purification and/or detection. For example, the polypeptides may be expressed as fusions with proteins such as maltose-binding protein (MBP), glutathione-S-transferase (GST), or thioredoxin (TRX). Kits for expression and purification of such fusion proteins are commercially available for New England BioLabs (Beverly, Mass.), Pharmacia (Piscataway, N.J.), and Invitrogen (Carlsbad Calif.), respectively. The polypeptides of the present invention can also be tagged with a small epitope and subsequently identified or purified using a specific antibody to the epitope. A preferred epitope is the FLAG epitope, which is commercially available from Eastman Kodak (New Haven, Conn.).

A signal sequence can be used to facilitate secretion and isolation of the secreted protein or other proteins of interest. Signal sequences are typically characterized by a core of hydrophobic amino acids that are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to polypeptides from which the signal sequence has been proteolytically cleaved (i.e., the cleavage products). In one embodiment, a polynucleotide sequence encoding a signal sequence can be operably linked in an expression vector to a protein of interest, such as a protein that is ordinarily not secreted or is otherwise difficult to isolate. The signal sequence directs secretion of the protein, such as from a eukaryotic host into which the expression vector is transformed, and the signal sequence is subsequently or concurrently cleaved. The protein can then be readily purified from the extracellular medium by art-recognized methods. Alternatively, the signal sequence can be linked to the protein of interest using a sequence that facilitates purification, such as with a GST domain.

In addition to the IBD marker polypeptides listed in Tables 2 or 5, and allelic variants and homologs thereof, the present invention also encompasses polypeptides that are structurally different from the polypeptides listed in Tables 2 or 5 (e.g., have a slightly altered sequence), but that have substantially the same biochemical properties as the disclosed polypeptides (e.g., are changed only in functionally nonessential amino acid residues). Such molecules include, but are not limited to, deliberately engineered variants containing alterations, substitutions, replacements, insertions, or deletions. Techniques and kits for such alterations, substitutions, replacements, insertions or deletions are well known to those skilled in the art.

The present invention also pertains to variants of the IBD differential marker proteins of the invention that function as either agonists or as antagonists to the marker proteins. In certain embodiments, an agonist of the marker proteins can retain substantially the same, or a subset, of the biological activities of the naturally occurring form of a marker protein or may enhance an activity of a marker protein. In certain embodiments, an antagonist of a marker protein can inhibit one or more of the activities of the naturally occurring form of the marker protein by, for example, competitively modulating an activity of a marker protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the marker protein. In another preferred embodiment, rhIL-11 and/or an agent that acts in a similar manner may serve as an agonist and an antagonist for IBD marker proteins of the invention depending on whether up- or downregulation of a particular IBD marker protein of interest is required for treatment of IBD.

Variants of the marker proteins can be generated by mutagenesis, e.g., discrete point mutation or truncation of a marker protein. Alternatively, variants of IBD marker proteins that function as either IBD marker protein agonists or as IBD marker protein antagonists can be identified by screening combinatorial libraries of mutants, e.g., truncation mutants, of an IBD marker protein for IBD marker protein agonist or antagonist activity. In one embodiment, a variegated library of IBD differential marker protein variants is generated by combinatorial mutagenesis at the polynucleotide level and is encoded by a variegated gene library. In certain embodiments, such protein may be used, for example, as a therapeutic protein of the invention. A variegated library of marker protein variants can be produced by, for example, enzymatically ligating a mixture of synthetic oligonucleotides into gene sequences such that a degenerate set of potential marker protein sequences is expressible as individual polypeptides, or alternatively, as a set of larger fusion proteins (e.g., for phage display) containing the set of marker protein sequences therein. There are a variety of methods that can be used to produce libraries of potential marker protein variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential marker protein sequences. Methods for synthesizing degenerate oligonucleotides are known in the art.

The polypeptides of the present invention may also be produced by known conventional chemical synthesis. Methods for chemically synthesizing the polypeptides of the present invention are well known to those skilled in the art. Such chemically synthetic polypeptides may possess biological properties in common with the natural, purified polypeptides, and thus may be employed as biologically active or immunological substitutes for the natural peptides.

Antibodies

In another aspect, the invention includes antibodies that are specific to proteins corresponding to, or encoded by, IBD differential markers of the invention. Preferably the antibodies are monoclonal, and most preferably, the antibodies are humanized, as per the description of antibodies described below. In one embodiment, antibodies to the protein encoded by the IBD marker Amy1 may be used in the invention. Other nonlimiting examples of antibodies that may be useful in the invention, include, but are not limited to, antibodies that immunospecifically bind to proteins encoded by the IBD markers Scya5 and RegIII.

Antibody molecules to the IBD marker polypeptides of the invention (anti-marker protein antibodies) may be produced by methods well known to those skilled in the art. For example, monoclonal antibodies can be produced by generation of hybridomas in accordance with known methods. Hybridomas formed in this manner are then screened using standard methods, such as enzyme-linked immunosorbent assay (ELISA), to identify one or more hybridomas that produce an antibody that specifically binds with the polypeptides of the present invention. A full-length polypeptide of the present invention may be used as the immunogen, or, alternatively, antigenic peptide fragments of the polypeptides may be used. An antigenic peptide of a polypeptide of the present invention comprises at least 7 continuous amino acid residues, and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

As an alternative to preparing monoclonal antibody-secreting hybridomas, a monoclonal antibody to a polypeptide of the present invention may be identified and isolated by screening a recombinant combinatorial immunoglobulin library (e.g., an antibody phage display library) with an IBD marker polypeptide of the present invention to thereby isolate immunoglobulin library members that bind to the polypeptides. Techniques and commercially available kits for generating and screening phage display libraries are well known to those skilled in the art. Additionally, examples of methods and reagents particularly amenable for use in generating and screening antibody display libraries can be found in the literature.

Polyclonal sera and antibodies may be produced by immunizing a suitable subject with a polypeptide of the present invention. The antibody titer in the immunized subject may be monitored over time by standard techniques, such as with ELISA using immobilized marker protein. If desired, the antibody molecules directed against a polypeptide of the present invention may be isolated from the subject or culture media and further purified by well-known techniques, such as protein A chromatography, to obtain an IgG fraction.

Additionally, recombinant anti-marker protein antibodies, such as chimeric, humanized, and single-chain antibodies, comprising both human and nonhuman portions, which can be made using standard recombinant DNA techniques, are within the scope of the invention. Humanized antibodies may also be produced using transgenic mice that are incapable of expressing endogenous immunoglobulin heavy and light chain genes, but that can express human heavy and light chain genes. Alternatively, humanized antibodies that recognize a selected epitope can be generated using a technique referred to as guided selection. In this approach, a selected nonhuman monoclonal antibody (e.g., a murine antibody) is used to guide the selection of a humanized antibody recognizing the same epitope.

Fragments of anti-marker antibodies may be produced by cleavage of the antibodies in accordance with methods well known in the art. For example, immunologically active F(ab′) and F(ab′)₂ fragments may be generated by treating the antibodies with an enzyme such as pepsin.

Anti-marker antibodies of the invention are also useful for isolating, purifying, and/or detecting IBD marker polypeptides in the supernatant, cellular lysate or on the cell surface. Antibodies disclosed in this invention can be used diagnostically to monitor levels of IBD marker proteins as part of a clinical testing procedure or targeting a therapeutic modulator to a cell or tissue comprising the antigen of the anti-marker antibody. For example, a therapeutic of the invention, including but not limited to a small molecule, can be linked to the anti-marker antibody in order to target the therapeutic to the cell or tissue expressing an IBD marker.

Screening

The IBD marker polynucleotides and polypeptides of the present invention may be used in screening assays to identify pharmacological agents, or lead compounds for agents, capable of modulating the activity of IBD markers and thus potentially capable of inhibiting IBD. Such screening assays are well known in the art. For example, samples from subjects diagnosed with or suspected of having IBD, or samples containing IBD markers (either natural or recombinant) can be contacted with one of a plurality of test compounds (e.g., small organic molecules, biological agents), and the activity of IBD differential markers in each of the treated samples can be compared to the activity of IBD differential markers in untreated samples or in samples contacted with different test compounds to determine whether any of the test compounds provides: 1) a substantially decreased level of expression or activity of IBD differential markers, thereby indicating an inhibitor of IBD differential marker activity, or 2) a substantially increased level of expression or activity of IBD differential markers, thereby indicating an activator of IBD differential marker activity. In a preferred embodiment, the identification of test compounds capable of modulating IBD differential marker activity is performed using high-throughput screening assays, such as provided by BIACORE® (Biacore International AB, Uppsala, Sweden), BRET (bioluminescence resonance energy transfer), and FRET (fluorescence resonance energy transfer) assays, as well as ELISA and cell-based assays.

In addition, the invention is further directed to a method of screening for test compounds capable of modulating the binding of an IBD differential marker listed in Table 2 to a binding partner, by combining the test compound, protein, and binding partner together and determining whether binding of the binding partner and protein occurs. As mentioned above, the bioactive agent may be any of a variety of naturally occurring or synthetic compounds, biomolecules, proteins, peptides, oligopeptides, polysaccharides, nucleotides or polynucleotides. The test compound may be either a small molecule or a bioactive agent. As discussed below, test compounds may be provided from a variety of libraries well known in the art.

The test compounds of the present invention may be obtained from any available source, including systematic libraries of natural and/or synthetic compounds. Test compounds may also be obtained by any of the numerous approaches in combinatorial library methods known in the art, including: biological libraries; peptoid libraries (libraries of molecules having the functionalities of peptides, but with a novel, nonpeptide backbones that are resistant to enzymatic degradation yet remain bioactive; see, e.g., Zuckermann et al. (1994) J. Med. Chem. 37:2678-85); spatially addressable parallel solid phase or solution phase libraries; synthetic library methods requiring deconvolution; the “one-bead, one-compound” library method; and synthetic library methods using affinity chromatography selection. The biological library and peptoid library approaches are limited to peptide libraries, while the other four approaches are applicable to peptide, nonpeptide oligomer or small molecule libraries of compounds (Lam (1997) Anticancer Drug Des. 12:145).

In a specific embodiment, the high-throughput screening assay detects the ability of a plurality of test compounds to bind to RT1.DMβ (or a homolog or ortholog thereof). In another specific embodiment, the high-throughput screening assay detects the ability of a plurality of test compounds to inhibit a RT1.DMβ binding partner (such as a ligand) to bind to RT1.DMβ. In another specific embodiment, the high-throughput screening assay detects the ability of a plurality of test compounds to modulate signaling through RegIII.

Methods for Diagnosing, Prognosing and Monitoring the Progress of Inflammatory Bowel Disease

The present invention provides methods for diagnosing, prognosing, and monitoring the progress of IBD in a subject that directly or indirectly results from aberrant expression or activity levels of IBD differential markers by detecting such aberrant expression or activity levels of IBD differential markers, including, but not limited to, the use of such methods in human subjects. For example, these methods may be performed by utilizing prepackaged diagnostic kits comprising at least one of the group comprising IBD differential marker polynucleotides and fragments thereof, IBD differential marker polypeptides and derivatives thereof, and modulators of IBD polynucleotides and/or polypeptides as described herein, which may be conveniently used, for example, in a clinical setting. In addition, one of skill in the art would recognize that changes in IBD differential markers could also be detected by other methods.

The diagnostic, prognostic, and monitoring assays of the present invention involve detecting and quantifying IBD differential marker gene products in biological samples. IBD differential marker gene products include, but are not limited to, IBD differential marker mRNAs, cDNAs and genomic DNAs and IBD differential marker poplypeptides; such gene products can be measured using methods well known to those skilled in the art.

For example, mRNA of IBD differential markers can be directly detected and quantified using hybridization-based assays, such as Northern hybridization, in situ hybridization, dot and slot blots, and oligonucleotide arrays (biochips). Hybridization-based assays refer to assays in which a probe nucleic acid is hybridized to a target nucleic acid. In some formats, the target, the probe, or both are immobilized. The immobilized nucleic acid may be DNA, RNA, or another oligonucleotide or polynucleotides, and may comprise naturally or nonnaturally occurring nucleotides, nucleotide analogs, or backbones. Methods of selecting nucleic acid probe sequences for use in the present invention are based on the nucleic acid sequences of the IBD differential markers and are well known in the art.

Alternatively, mRNA of IBD differential markers can be amplified before detection and quantitation. Such amplification-based assays are well known in the art and include polymerase chain reaction (PCR), reverse-transcription-PCR (RT-PCR), PCR-enzyme-linked immunosorbent assay (PCR-ELISA), ligase chain reaction (LCR), self-sustained sequence replication, transcriptional amplification system, Q-beta Replicase or any other polynucleotide amplification method. Primers and probes for producing and detecting amplified IBD differential gene products can be readily designed and produced without undue experimentation by those of skill in the art based on the nucleic acid sequences of the IBD differential markers listed in Tables 2 or 5. Amplified IBD differential gene products may be directly analyzed, for example, by gel electrophoresis; by hybridization to a probe nucleic acid; by sequencing; by detection of a fluorescent, phosphorescent, or radioactive signal; or by any of a variety of well-known methods. In addition, methods are known to those of skill in the art for increasing the signal produced by amplification of target nucleic acid sequences. One of skill in the art will recognize that whichever amplification method is used, a variety of quantitative methods known in the art (e.g., quantitative PCR) may be used if quantitation of IBD differential gene products is desired.

IBD differential marker polypeptides of the invention (or fragments thereof) can be detected using various well-known immunological assays employing anti-marker antibodies described above. Immunological assays refer to assays that utilize an antibody (e.g., polyclonal, monoclonal, chimeric, humanized, scFv and fragments thereof) that specifically binds to an IBD differential polypeptide (or fragment thereof). Such well-known immunological assays suitable for the practice of the present invention include ELISA, radioimmunoassay (RIA), immunoprecipitation, immunofluorescence, fluorescence-activated cell sorting (FACS) and Western blotting. In addition, the antibody can be labeled with a radioactive marker whose presence and location in a subject can be detected by standard imaging techniques.

Each marker may be considered individually, although it is within the scope of the invention to provide combinations of two or more markers for use in the methods and compositions of the invention to increase the confidence of the analysis. In another aspect, the invention provides panels of the IBD differential markers of the invention. A panel of markers comprises 2 or more IBD differential markers. A panel may also comprise 2-5, 5-15, 15-35, 35-50, 50-100, or more than 100 IBD differential markers. In a preferred embodiment, these panels of markers are selected such that the markers within any one panel share certain features. For example, the markers of a first panel may each exhibit at least a two-fold increase in quantity or activity in an IBD sample, as compared to a sample that is substantially free of IBD from the same subject or a sample that is substantially free of IBD from a different subject without IBD. Alternatively, markers of a second panel may each exhibit differential regulation as compared to a first panel. Similarly, different panels of markers may be composed of markers from different functional categories, or samples (e.g., kidney, spleen, node, brain, intestine, colon, heart or urine), or may be selected to represent different stages of IBD. Panels of the IBD differential markers of the invention may be made by independently selecting markers from Table 2. In another embodiment, the panel of markers may be made by independently selecting markers from Table 5.

In addition to providing panels of IBD differential markers, it is within the scope of the invention to provide a panel of IBD differential markers conveniently coupled to a solid support. For example, IBD differential marker polynucleotides of the invention may be coupled to an array (e.g., a biochip for hybridization analysis), to a resin (e.g., a resin that can be packed into a column for column chromatography), or a matrix (e.g., a nitrocellulose matrix for Northern blot analysis) using well-known methods in the art. By providing such support, discrete analysis of the presence or activity in a sample of each marker selected for the panel can be detected. For example, in an array, polynucleotides complementary to each member of a panel of markers may be individually attached to different known locations on the array using methods well known in the art. The array may be hybridized with, for example, polynucleotides extracted from a blood or colon sample from a subject. The hybridization of polynucleotides from the sample with the array at any location on the array can be detected, and thus the presence or quantity of the marker in the sample can be ascertained. Thus, not only tissue specificity, but also the level of expression of a panel of IBD markers in the tissue is ascertainable. In a preferred embodiment, an array based on a biochip is employed. Similarly, ELISA analyses may be performed on immobilized antibodies specific for different polypeptide markers hybridized to a protein sample from a subject.

“Diagnostic” or “diagnosing” means identifying the presence or absence of a pathologic condition. Diagnostic methods involve detecting aberrant expression of IBD differential markers by determining a test amount of IBD differential marker gene products (e.g., mRNA, cDNA, or polypeptide, including fragments thereof) in a biological sample from a subject (human or nonhuman mammal), and comparing the test amount with the normal amount or range (i.e., an amount or range from an individual(s) known not to suffer from IBD) for the IBD differential marker gene product.

In one embodiment, the levels of IBD markers in the two samples are compared, and a modulation in one or more IBD markers in the test sample indicates IBD. In other embodiments the modulation of 2, 3, 4 or more markers indicates a severe case of IBD. In another aspect, the invention provides markers whose quantity or activity is correlated with different manifestations or severity or types of IBD. The subsequent level of expression may further be compared to different expression profiles of various stages of the disorder to confirm whether the subject has a matching profile. Although a particular diagnostic method may not provide a definitive diagnosis of IBD, it suffices if the method provides a positive indication that aids in diagnosis.

The present invention also provides methods for prognosing IBD by detecting aberrant expression or activity levels of IBD differential markers. “Prognostic” or “prognosing” means predicting the probable development and/or severity of a pathologic condition. Prognostic methods involve determining the test amount of an IBD differential marker gene product in a biological sample from a subject, and comparing the test amount to a prognostic amount or range (i.e., an amount or range from individuals with varying severities of IBD) for the IBD differential gene product. Various amounts of the IBD differential gene product in a test sample are consistent with certain prognoses for IBD. The detection of an amount of IBD differential gene product at a particular prognostic level provides for a prognosis for the subject. In one embodiment of the present invention, as related to IBD, aberrant expression or activity of upregulated IBD markers is typically correlated with an abnormal increase. In another embodiment of the present invention, as related to IBD, aberrant expression or activity of downregulated IBD markers is typically correlated with an abnormal decrease.

In addition, the prognostic assays described herein can be used to determine whether a subject can be administered an agent (e.g., an agonist, antagonist, peptidomimetic, protein, peptide, polynucleotide, small molecule, or other drug candidate) to treat or prevent IBD associated with aberrant marker expression or activity, such as, for example, rhIL-11.

For example, the IBD differential marker designated Scya5 (group 2, Table 2) has increased expression in HLA-B27 rat tissue samples, relative to control Fischer 344 rat tissue samples. The presence of increased mRNA for this marker or the human homolog thereof (or other upregulated IBD markers listed in Table 2 or human homologs thereof), or increased levels of the protein products of this marker or the human homolog thereof (and other upregulated IBD markers set forth in Table 2, or human homologs thereof) serve as markers for IBD. Accordingly, modulation of upregulated IBD markers, such as Scya5, to normal levels (e.g., levels similar or substantially similar to tissue substantially free of IBD) as compared to Fischer 344 rat tissue may allow for amelioration of IBD. Preferably, for the purposes of the present invention, increased levels of the upregulated IBD markers of the invention are increased by an abnormal magnitude, wherein the level of expression is outside the standard deviation for the same marker as compared to Fischer 344 rat tissue. Most preferably, the upregulated IBD marker is enhanced or increased relative to Fischer 344 rat tissue samples by at least 2-, 3-, or 4-fold or more. Alternatively, the upregulated IBD marker is modulated to be similar to a control sample that is taken from a subject (human or otherwise) or tissue that is substantially free of IBD. In one embodiment, an upregulated IBD marker listed in Table 2 is returned to near normal levels upon treatment with rhIL-11, as shown in Table 4. For example, the transcription factor Hmgiy (group 5, Table 2) is upregulated by a factor of 2.4667 in the HLA-B27 rat tissue, as shown in Table 2. Upon treatment of HLA-B27 tissue with rhIL-11, Hmgiy expression is downregulated by a factor of 2.53 (see Table 4), thereby approximating the normal level of gene expression and indicating that rhIL-11 was efficacious for treating IBD. One of skill in the art will appreciate the application of such control samples.

As another example, the gene designated Amy1 (group 13, Table 2) has decreased expression in HLA-B27 rat tissue samples relative to Fischer 344 rat tissue samples. The presence of decreased mRNA for this marker (and for other downregulated IBD markers set forth in Table 2, or human homologs thereof), or decreased levels of the protein products of this gene (and for other down-regulated IBD markers set forth in Table 2, or human homologs thereof) serve as markers for IBD. Accordingly, modulation of downregulated IBD markers to normal levels (e.g., levels similar or substantially similar to tissue substantially free of IBD) as compared to Fischer 344 rat tissue may allow for amelioration of IBD. Preferably for the purposes of the present invention, decreased levels of the down-regulated IBD markers of the invention are decreased by an abnormal magnitude, wherein the level of expression is outside the standard deviation for the same marker as compared to HLA-B27 rat tissue. Most preferably the marker is decreased relative to control samples by at least 2-, 3- or 4-fold or more. Alternatively, the downregulated IBD marker is modulated to be similar to a control sample that is taken from a subject, tissue or cell that is substantially free of IBD. For example, the gene Prss1 (group 14, Table 2), which is involved in protein metabolism, is downregulated in HLA-B27 rat tissue by a factor of 21.64 (Table 2). Upon treatment of the HLA-B27 rat tissue with rhIL-11, the expression of Prss1 was increased by a factor of 35.93 (Table 4), thereby indicating that treatment of HLA-B27 rat tissue with rhIL-11 was efficacious for treating IBD. One of skill in the art will appreciate the application of such control samples.

In relation to the field of gastroenterology, prognostic assays can be devised to determine whether a subject undergoing treatment for such disorder has a poor outlook for long-term survival or disease progression. In a preferred embodiment, prognosis can be determined shortly after diagnosis, i.e., within a few days. By establishing expression profiles of different stages of IBD, from onset to acute disease, an expression pattern may emerge to correlate a particular expression profile to increased likelihood of a poor prognosis. The prognosis may then be used to devise a more aggressive treatment program to avert chronic IBD and enhance the likelihood of long-term survival and well-being.

In a preferred embodiment of the invention, the disclosed molecules and methods are used on a biological sample to detect, in IBD differential marker genes, the presence of one or more genetic alterations well known to result in aberrant expression of IBD differential markers. Such detecting can be used to determine the severity of IBD or to prognosticate the potential for IBD due to aberrant expression or activity of IBD markers. In a further specific embodiment, one or more genetic alterations are correlated with the prognosis or susceptibility of a subject to IBD. Genetic alterations in an IBD differential marker gene from a sample can be identified by well-known methods in the art, including, but not limited to, sequencing reactions, electrophoretic mobility assays, and oligonucleotide hybridizations. For example, if a mutation is detected in a Scya5 polynucleotide or Scya5 polypeptide that results in aberrant Scya5 activity associated with IBD, such Scya5 mutation is correlated with the prognosis or susceptibility of a subject to IBD, including ulcerative colitis, Irritable Bowel Syndrome and Crohn's disease (regional enteritis).

The present invention also provides methods for monitoring the progress or course of IBD by monitoring the expression or activity of IBD markers. Monitoring methods involve determining the test amount of an IBD marker gene product in biological samples taken from a subject at a first and second time, and comparing the amounts. A change in the amount of an IBD marker, or changes in the amounts of IBD markers, between the first and second time indicates a change in the course of IBD. Such monitoring assays are also useful for evaluating the efficacy of a particular therapeutic intervention in patients during clinical trials, i.e., evaluating the modulation of IBD markers in response to therapeutic agents provided herein.

It will be appreciated that the assay methods of the present invention do not necessarily require measurement of absolute values of IBD differential marker gene products because relative values are sufficient for many applications of these methods. It will also be appreciated that in addition to the quantity or abundance of IBD differential gene products, variant or abnormal IBD gene products or their expression patterns (e.g., mutated transcripts, truncated polypeptides) may be identified by comparison to normal gene products and expression patterns.

Expression levels of IBD markers in methods outlined above can be detected in a variety of biological samples, including tissues, cells and biological fluid in which an IBD differential marker is expressed (e.g., a colon biopsy). Biological samples include those taken within subject (i.e., in vivo) and those taken from a subject (i.e., in vitro). Preferably, expression levels of IBD markers in methods outlined above are detected from the duodenum, ileum, jejunum, colon and rectum. Additionally, expression levels of IBD markers can be detected from feces.

Methods of Treatment

The present invention provides for both prophylactic and therapeutic methods of treating a subject at risk for, susceptible to or diagnosed with IBD. Subjects at risk, susceptible to or diagnosed with IBD that is caused or contributed to by aberrant marker expression or activity can be identified by, for example, any or a combination of diagnostic or prognostic assays as described herein. In one aspect, the invention provides prophylactic methods for preventing, in a subject, IBD associated with aberrant IBD differential marker expression or activity, by administering to the subject a marker protein or an agent, which modulates marker protein expression or activity. Administration of a prophylactic agent can occur prior to the manifestation of symptoms characteristic of the differential marker protein expression, such that IBD is prevented or, alternatively, delayed in its progression. Another aspect of the invention pertains to therapeutic methods of modulating expression or activity levels of IBD markers for therapeutic purposes. Accordingly, in an exemplary embodiment, the modulatory method of the invention involves contacting a cell with an agent that modulates one or more of the activities of IBD markers associated with the cell.

An agent that modulates expression or activity levels of IBD markers activity can be an agent as described herein, such as an IBD marker polynucleotide or protein, a naturally occurring target molecule of an IBD marker protein (e.g., a marker protein substrate), an anti-marker protein antibody, an IBD marker modulator (e.g., agonist or antagonist), or other small molecule. The appropriate agent can be determined based on screening assays described herein.

These modulatory methods can be performed in vitro (e.g., by culturing the cell with the agent) or, alternatively, in vivo (e.g., by administering the agent to a subject). In one embodiment, the method involves administering a marker protein or polynucleotide molecule as therapy to compensate for reduced or aberrant marker protein expression or activity. Stimulation of marker protein activity is desirable in situations in which marker protein is abnormally downregulated and/or in which increased marker protein activity is likely to have a beneficial effect. Likewise, particularly with regard to the markers listed in Tables 2 or 4, which are differentially expressed in HLA-B27 rat cells, alteration of IBD marker protein or activity to levels similar to Fischer 344 rat cells is likely to have a beneficial effect with respect to IBD.

For example the IBD differential marker Amy1 (group 13, Table 2) is abnormally decreased in activity or expression levels in a subject diagnosed with or suspected of having IBD. In this embodiment, treatment of such a subject may comprise administering an agonist wherein such agonist provides increased activity or expression of Amy1. In this embodiment, treatment of such a subject may comprise administering an agent with an effect similar to that of rhIL-11, which may provide increased activity or expression of Amy1 (e.g., Amy1 is decreased by a factor of 126.9 in diseased tissue (Table 2), but increased by a factor of 185.92 after treatment with rhIL-11 (Table 4)).

As another nonlimiting example, the IBD differential marker Scya5 (group 2, Table 2) is abnormally increased in activity or expression levels in a subject diagnosed with or suspected of having IBD (e.g., Scya5 expression increased by a factor of 14.9 in diseased tissue (Table 2)); alternatively, a decreased expression of normal levels of Scya5 is desired. In these embodiments, treatment of such a subject may comprise administering an antagonist wherein such antagonist provides decreased activity or expression of Scya5.

In another embodiment of the invention, the IBD differential marker is modulated in diseased tissue upon treatment of rhIL-11, such as, for example, RegIII (Table 5). In this embodiment, treatment of a subject may comprise administering an agent with an effect similar to that of rhIL-11 to increase the level of expression of RegIII (expression increased by a factor of 31.00 upon treatment of diseased tissue with rhIL-11 (Table 5)).

In a specific embodiment, a protein therapeutic of the invention may comprise a soluble RegIII-ligand protein. Administration of such a therapeutic may induce suppressive bioactivity, and therefore may be used to ameliorate IBD. In another example, an inhibitory agent is an antisense RT1.DMβ (or homolog thereof) polynucleotide.

Of great interest are four genes that were upregulated in the colon of the rhIL-11-treated rat and were not identified as disease-related. Two of these genes, RegI and TFF2, are known in the art to be associated with IBD (Lawrance et al. (2001) Hum. Mol. Genet. 10:445-56; Thim et al., International Pat. Appln. Publication No. WO 02/46226). The other two genes, RegIII and Ins2, are genes of the invention and are listed in Table 5. All four genes encode known or putative growth factors of intestinal epithelial cells, and all of these growth factors are secreted proteins. The expression, or upregulation of the expression, of these four genes appears to be involved in the healing process brought about by treatment of IBD with rhIL-1. Thus, the genes and proteins of the invention (RegIII and Ins2), offer great potential as biotherapeutics to treat intestinal epithelial damage associated with IBD. One of skill in the art will recognize the value of including RegI and/or TFF2 in any biotherapeutic treatment involving RegIII and/or Ins2.

Several pharmacogenomic approaches to be considered in determining whether to administer an IBD differential marker are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous, subcutaneous, intramuscular), oral (e.g., inhalation), transdermal (topical), transmucosal, and rectal administration. The pharmaceutical compositions compatible with each intended route are well known in the art.

Modifications to the above-described compositions and methods of the invention, according to standard techniques, will be readily apparent to one skilled in the art and are meant to be encompassed by the invention. This invention is further illustrated by the following examples that should not be construed as limiting. The contents of all references, patents and published patent applications cited throughout this application, as well as in the Figures and Tables, are incorporated herein by reference.

### EXAMPLES

### Example 1

Pharmacogenomic analyses of the effects of rhIL-11 in the HLA-B27 rat were studied. Gene expression profiles of inflamed colons of HLA-B27 rats were compared to rhIL-11-treated HLA-B27 rats and uninflamed controls. One hundred and seventy-one differentially expressed genes were identified in the diseased colon, many of which are associated with metabolism. rhIL-11 treatment was associated with amelioration of disease and returned the levels of 27 of these disease-related genes to normal levels. rhIL-11 treatment also significantly induced the expression in colonic epithelial cells of four intestinal growth factor (or putative growth factor) genes that were not differentially expressed in diseased colon: RegI, RegIII, Insulin II and TFF2. Pulse-chase experiments with BrdU indicated that rhIL-11 treatment significantly expanded the proliferation of intestinal epithelial cells. These results show that rhIL-11 treatment is associated with the expression of epithelial cell growth factors, epithelial cell proliferation, and the restitution of normal gene expression levels for metabolic enzymes.

### Example 1.1

### Experimental Design

Male transgenic rats engineered to overexpress the human MHC Class I allele HLA-B27 and β2-microglobulin genes on a Fischer 344 background were obtained from Taconic (Germantown, N.Y.). Rats in this study were between the ages of 22 to 28 weeks when IBD was evident based on the presence of diarrhea or soft stool character. Aged-matched male Fischer 344 rats were also obtained from Taconic. rhIL-11 (specific activity 1.5×10⁶ U/mg) was manufactured at Genetics Institute. Rats received 37.5 μg/kg rhIL-11 or vehicle subcutaneously at time 0 and 48 h. At the time of the second dose, both the vehicle and rhIL-11-treated groups received an intraperitoneal injection of 500 μg/kg BrdU (Sigma, St. Louis). Three vehicle-treated HLA-B27 rats and five rhIL-11-treated HLA-B27 rats were killed 4 and 24 hr after the second dose of rhIL-11/BrdU. Fischer 344 rats received 37.5 μg/kg rhIL-11 or vehicle subcutaneously at time 0 and 48 hr, and an intraperitoneal injection of 500 μg/kg BrdU at the second time point (48 hr). Five rhIL-11-treated Fischer 344 rats and 5 vehicle-treated rats were sacrificed 4 hr after the vehicle/BrdU or rhIL-11/BrdU dose. These animals represented normal, or nondiseased tissue. The HLA-B27 rats were observed daily for stool character, which was characterized as normal, soft or diarrhea.

After the rats were killed, sections of the colon were removed in a standardized manner known in the art to insure that the same regions of the colon were represented in both the gene expression and histological analysis (Keith et al. (1994) Stem Cells 12 (suppl. 1):79-90; Peterson et al. (1998) supra). Tissue obtained for histological, immunohistological and in situ analysis was rinsed in ice-cold phosphate-buffered saline (PBS) then fixed for 24 hours in 10% neutral-buffered formalin. The remaining portion of the tissue was rinsed in ice cold PBS and snap frozen in liquid nitrogen for use in RNA preparation.

### Example 1.2

### RNA Extraction

Individual colonic tissue from individual rats was pulverized using a mortar and pestle cooled in liquid nitrogen. Total RNA was prepared using the RNAgent Total RNA Isolation™ kit (Promega, Madison, Wis.) following the manufacturer's protocol.

### Example 1.3

### cRNA Probe Generation for In Situ Hybridization

Templates to generate in situ hybridization probes were amplified from colonic RNA isolated from a rhIL-1-treated HLA-B27 rat using RT-PCR. Total RNA was DNAse treated for 30 min at 37° C. using 10 Unit/ml RQ1 RNAse-free DNAse (Promega) to remove contaminating DNA. The DNAse was removed and the total RNA cleaned-up by passing the sample through an RNeasy™ spin column (Qiagen, Valencia, Calif.) according to the manufacturer's protocol. Total RNA (1 μg) was reverse transcribed (RT) using the GeneAmp RT-PCR™ kit (Perkin Elmer, Norwalk, Conn.) and random hexamers according to the manufacturer's protocol. One tenth of the RT reaction volume was subjected to 40 cycles of amplification using gene-specific oligos (described below) and the Optimized Buffer C™ kit (Invitrogen, Carlsbad, Calif.). The 5′ primers for each gene included the addition of a T3 bacteriophage RNA polymerase recognition sequence immediately upstream (5′) of gene-specific sequences; the 3′ primers similarly included a T7 bacteriophage RNA polymerase recognition sequence immediately upstream (5′) of gene-specific sequences. Primers sequences used to amplify each gene are listed below.

| | |
|---|---|
| RegI forward | |
| (5′ to 3′ orientation) | |
| GCGCGCAATTAACCCTCACTAAAGGGAGATAACAGT | (SEQ ID NO. 1) |
| TGTGATGCC | |
| RegI reverse | |
| (5′ to 3′ orientation) | |
| ATGGATTAATACGACTCACTATAGGGTTTATTTAAA | (SEQ ID NO. 2) |
| TGTGCAGGGTT | |
| RegIII forward | |
| (5′ to 3′ orientation) | |
| GCGCGCAATTAACCCTCACTAAAGGGAAGGTCACCG | (SEQ ID NO. 3) |
| TGACAAGG | |
| RegIII reverse | |
| (5′ to 3′ orientation) | |
| ATGGATTAATACGACTCACTATAGGGCAAGATTGCA | (SEQ ID NO. 4) |
| AAGCAGGAACT | |
| TFF2 forward | |
| (5′ to 3′ orientation) | |
| GCGCGCAATTAACCCTCACTAAAGGGATCTTCGAAG | (SEQ ID NO. 5) |
| TGCCCTGG | |
| TFF2 reverse | |
| (5′ to 3′ orientation) | |
| ATGGATTAATACGACTCACTATAGGGCCACTGCTGA | (SEQ ID NO. 6) |
| GGCTCAAGAGA | |
| Insulin II forward | |
| (5′ to 3′ orientation) | |
| GCGCGCAATTAACCCTCACTAAAGGGACCCACAAGT | (SEQ ID NO. 7) |
| GGCA | |
| Insulin II reverse | |
| (5′ to 3′ orientation) | |
| ATGGATTAATACGACTCACTATAGGGTTGCAGTAGT | (SEQ ID NO. 8) |
| TCTCCAGTTGG | |

Amplified DNA fragments were purified from 4% agarose gels using the Qiaquick Gel™ extraction kit (Qiagen) and used as template to generate sense and antisense cRNA probes using the Maxiscript T3/T7™ kit (Ambion, Austin, Tex.) and Digoxigenin-11-uridine-5′-triphosphate (Roche Diagnostics, Indianapolis, Ind.) as the labeling nucleotide.

### Example 1.4

### In Situ Hybridization

Sections of paraffin embedded tissue were deparaffinized in xylene, 2 times, for 3 minutes each, then rehydrated in water. Following a rinse in RNAse-free water and phosphate buffered saline (PBS), permeabilization was performed by incubation with 0.2% Triton-X 100/PBS for 15 minutes. The sections were washed 2× in PBS, 3 minutes, then subjected to proteinase K (PK) (Sigma, St. Louis, Mo.) digestion in 0.1M Tris and 50 mM EDTA (pH 8.0) prewarmed at 37° C. containing 5 mg/mL PK for 15 minutes. PK digestions were stopped by washing with 0.1M glycine/PBS for 5 minutes followed by post-fixation with 4% paraformaldehyde/PBS for 3 minutes and a PBS rinse. To prevent nonspecific electrostatic binding of the probe, sections were immersed in 0.25% acetic anhydride and 0.1M triethanolamine solution (pH 8.0) for 10 minutes, followed by 15 seconds in 20% acetic acid at 4° C. After 3 changes in PBS, 5 minutes each, sections were dehydrated through 70%, 90% and 100% ethanol, each at 3 minutes. The sections were completely air-dried. Forty ml of prehybridization buffer containing 55% deionized formamide, 5.5× saline sodium citrate (SSC), 110 mg/ml dextran sulfate, 0.55% lauryl sulfate (SDS) and 100 ug/ml herring sperm DNA was applied to the slides and incubated at 52° C. for 30 minutes to reduce nonspecific binding. Forty ml of hybridization buffer containing 5 ng/ml of digoxigenin-labeled probes was applied to each section and the slides were incubated overnight at 52° C.

Following hybridization the sections were immersed in 2×SSC/0.1% SDS at room temperature, 4 changes, 5 minutes each. To ensure specific binding of the probe, sections were washed in high stringency solution containing 0.1×SSC/0.1% SDS at 52° C., 2 changes, 10 minutes each. The labeled probe was detected with anti-digoxigenin antibody conjugated to alkaline phosphatase complex (Roche Diagnostics) diluted 1:100 in 2% normal sheep serum/0.1% Triton X-100. Labeled probe was developed with 5-Bromo-4-Chloro-3-Indoxyl Phosphate, Nitro Blue Tetrazolium Chloride and Iodonitrotetrazolium Violet (BCIP/NBT/INT) (Dako, Carpinteria, Calif.), washed in water, stained briefly with hematoxylin and mounted in aqueous mountant before microscopic examination.

### Example 1.5

### Histological Evaluation

Hematoxylin and eosin stained 5 □M tissue sections were evaluated without knowledge of treatment group and scored using a scale modified after Boughton-Smith (Peterson et al. (1998) supra; Boughton-Smith et al. (1998) Br. J. Pharmacol. 94:65-72; Greenwood-Van Meerveld et al. (2000) Lab. Invest. 80:1269-80). After scoring, the samples were unblinded and data was combined and tabulated, and then analyzed by ANOVA linear modeling (Abacus Concepts, Berkeley, Calif.) with multiple mean comparisons. Differences between the group means were considered significant if p<0.05.

### Example 1.6

### Histology Scoring System

The following Boughton-Smith histology scoring system was used for histological evaluation (Boughton-Smith et al. (1998) Br. J. Pharmacol. 94:65-72).

| Criteria | Severity | Score |
|---|---|---|
| Ulceration | No ulcer, epithelization | 0 |
| | Small ulcers | 1 |
| | Large Ulcers | 2 |
| Inflammation | None | 0 |
| | Mild | 1 |
| | Moderate | 2 |
| | Severe | 3 |
| Depth of Lesion | None | 0 |
| | Submucosa | 1 |
| | Muscularis propria | 2 |
| | Serosa | 3 |
| Fibrosis | None | 0 |
| | Mild | 1 |
| | Severe | 2 |

### Example 1.7

### BrdU Immunohistochemistry

Five μm sections of colonic tissues from rhIL-11- and vehicle-treated rats were deparaffinized in xylene and rehydrated through a graded series of ethanol to water. The slides were denatured in 2N hydrochloric acid for 30 min at room temperature. Following a PBS wash, the sections were proteinase K treated in 0.125% (w/v) PK (Sigma) for 5 min at room temperature. Next, the slides were stained using a Ventana TechMate 500™ automated immunostainer (Ventana Medical Systems Inc., Tucson, Ariz.). Sections were incubated with either anti-BrdU antibody (Becton Dickenson, San Jose, Calif.) or an appropriate isotype control at 0.1 mg/mL for 1 hr at room temperature. A biotinylated anti-mouse IgG antibody (Vector Laboratories, Inc., Burlingame, Calif.) was used as the secondary antibody and was incubated for 30 min at room temperature followed by incubation with a streptavidin-peroxidase linker (Signet Pathology Systems, Dedham, Mass.) for 25 minute. Sections were incubated with the chromogen Nova Red (Vector Laboratories, Inc., Burlingame, Calif.), counterstained with hematoxylin, and dehydrated in a graded series of ethanol to xylene. The slides were cover slipped with a synthetic mount media (Permount, Fischer Scientific, Inc) and evaluated using a Nikon Eclipse E400 microscope.

Sections were analyzed for the presence and quantification of BrdU positive cells by counting five crypts per slide and calculating the percentage of BrdU positive cells/total number of epithelial cells (FIG. 2). Data was analyzed by ANOVA and Tukey's multiple comparison test, using GraphPad Prism™ software (GraphPad Software, Inc. San Diego, Calif.).

### Example 1.8

### Hybridization of cRNA to Oligonucleotide Array

The Affymetrix RG_U34A rat GeneChip® (Affymetrix, Santa Clara, Calif.) was used in expression profile studies. The RG_U34A chip contains probes derived from all full-length or annotated rat sequence from Build #34 of the UniGene™ Database (created from GenBank 107/dbEST Nov. 18, 1998) and supplemented with additional annotated gene sequences from GenBank 110 as well as EST sequences (www.affymetrix.com).

Total RNA (10 μg) was converted to biotinylated cRNA according to the Affymetrix protocol. Complementary DNA (cDNA) was produced by priming the total RNA with an oligo-dT primer containing a T7 polymerase promoter sequence on the 5′end and reverse transcribed with 200 units of Superscript RT II™ (Gibco BRL, Gaithersburg, Md.) at 56° C. for 1 hour in 1× first strand buffer and 0.5 mM each dNTP (Gibco BRL). Second strand synthesis was performed by the addition of 40 units DNA Pol I, 10 units E. coli DNA ligases, 2 units RNase H, 30 ml second strand buffer (Gibco BRL), 3 ml of 10 mM dNTP (2.5 mM each) and dH20 to 150 ml final volume and incubated at 15° C. for 2 hours. The cDNA was used as template for in vitro transcription using a T7 RNA polymerase kit (Ambion, Woodland Hills, Tex.). Eleven control transcripts ranging in abundance from 1:300,000 (or 3 ppm) to 1:100 (or 100 ppm) were spiked into each sample prior to the in vitro transcription reaction to act as a standard curve used to normalize hybridization data between chips (Hill et al. (2000) Science 290:809-12). The biotinylated cRNA was purified using a RNeasy spin column (Qiagen) and quantitated using a spectrophotometer. Labeled cRNA (15 μg) was fragmented in a 40 μl volume containing 40 mM Tris-acetate pH 8.0, 100 nM KOAc, 30 nM MgOAc for 35 min at 94° C. The fragmented cRNA was diluted in 1×MES buffer containing 100 μg/ml herring sperm DNA and 50 μg/ml acetylated BSA (Gibco) and denatured for 5 min at 99° C. followed immediately by 5 min at 45° C. Insoluble material was removed by a brief centrifugation and the hybridization mix was added to each array and incubated at 45° C. for 16 hr with continuous rotation at 60 rpm. After incubation, the hybridization mix was removed and the chips were extensively washed with 6×SSPET as described in the Affymetrix protocol.

### Example 1.9

### GeneChip® Data Analysis

The raw fluorescent intensity value of each gene was measured at a resolution of 6 μm with a Hewlett-Packard Gene Array Scanner. GeneChip® software 3.2 (Affymetrix), which uses an algorithm to determine if a gene is “present” or “absent” as well as the specific hybridization intensity values or “average differences” of each gene on the array, was used to evaluate the fluorescent data. The average difference for each gene was normalized to frequency values by referral to the average differences of the 11 control transcripts of known abundance that were spiked into each hybridization mix according to the procedure of Hill et al. (2000) Science 290:809-12. The frequency of each gene was calculated and represents a value equal to the total number of individual gene transcripts per 10⁶ total transcripts.

The frequency of each gene was evaluated and the gene was included in the study if it met the following criteria. First, genes that were called “present” by the GeneChip® software in at least 60% of the arrays comprising one or more groups were included in the analysis (3365 genes met this criteria in the Fischer 344 vs. HLA-B27 rat comparison and 3064 met this criteria in the HLA-B27 vs. rhIL-11-treated HLA-B27 rat comparison). Second, for comparison between treatment groups, a t-test was applied to identify the subset of genes that had a significant (p<0.05) increase or decrease in frequency values. Third, average-fold changes in frequency values across the statistically significant subset of genes were required to be 2.4-fold or greater. Fourth, frequency values for a gene considered to be statistically significant were required to be above 10 in 60% of the animals in one or more groups (171 genes met this criteria in the Fischer 344 vs. HLA-B27 rat comparison and 35 genes met this criteria in the HLA-B27 vs. rhIL-11-treated HLA-B27 rat comparison). These criteria were established based upon replicate experiments that estimated the intra-array reproducibility.

### Example 1.10

### Using LocusLink™ and Unigene™ to Assign Gene Content to Probe Sets on the RG U34A Affymetrix GeneChip®

LocusLink™ is a compendium of gene sequences submitted to GenBank that are representative of the same gene in five species (orthologous sequences). This grouping and classification provides a single query interface to curated sequences providing descriptive information about genetic function and loci. One of the current limitations of the LocusLink™ gene collection is the relatively few number of rat genes that have been curated compared to mouse and human. There are approximately 3800 genes classified in rats, whereas there are approximately 22,500 and 33,000 genes classified for humans and mice, respectively. To supplement the available gene information for rats, we used a simple BLAST screen to acquire gene information for the RG_U34A rat GeneChip® array from orthologous sequences present on the human HuGeneFL, and murine Mu11KsubA and Mu11KsubB Affymetrix GeneChip® arrays.

For each probe set on an array, there is a corresponding target sequence, the specific portion of a complete sequence record from which oligo probes used for gene expression are selected. Target sequences were collected for each array and assembled into species-specific sets. In addition, the complete sequence records relating to the target sequences for these arrays were also collected and assembled into species-specific sets. A BLASTN search was performed for each target sequence against each of the two complete sequence collections from dissimilar species to assist in identifying orthologs. All target sequences from the RG_U34A array were BLASTed against the complete sequence collections for both the mouse and human arrays identified above. The results were then used to provide a quick screen for orthologous sequences.

Given a target sequence on the RG_U34A array, the top BLAST hit from the mouse complete sequence collection was identified. The BLAST result for the target sequence of this top hit against the rat complete sequence set was then examined. If the top BLAST hit of this search yielded the complete sequence related to the original rat target sequence, this was identified as a reciprocal BLAST hit and given an appropriate evidence score. Additionally, an evidence score was assigned based on the e-value of the original BLAST result. This procedure was also performed against the human complete sequence collection. The result of these screens was a summed evidence score. If the score for an associated human or mouse sequence was of sufficient value, the rat sequence was identified as being orthologous to that human or mouse sequence, and gene content information was shared amongst the orthologous sequences. If the evidence score was not of sufficient value, more involved sequence analysis was performed to attempt to identify orthologous sequences.

### Example 1.11

### Results: Identification of Disease-Related Gene Expression

To identify disease-related genes that are differentially expressed in the inflamed colon of the HLA-B27 rat model of IBD, we compared the gene expression profile of RNA isolated from the diseased colon of HLA-B27 rats with that of the nondiseased colon of the Fischer 344 rat. The expression profile of 5 HLA-B27 colons and 5 Fischer 344 colons was determined for individual animals using the RG_U34A, Affymetrix Rat U34A GeneChip® (total of 10 chips), which is capable of analyzing the expression of 8800 genes. The analysis software (EPIKS Explorer, Genetics Institute) yielded an absolute frequency value and a “present,” “absent” or “marginal” detection call for each gene. The data was reduced according to criteria set forth in above. One hundred and seventy-one genes were identified as differentially expressed in the diseased colon. Expression levels of 89 genes are upregulated in disease and 82 genes are down-regulated compared to a nondiseased colon (Table 1). The majority of the gene expression level changes were at the magnitude of 2.4- to 5.0-fold. By far the most robust differential gene expression changes occurred in genes involved in protein, lipid and carbohydrate metabolism that were downregulated in the diseased colon. Twelve genes were upregulated higher than 5.0-fold compared to the nondiseased colon while 36 genes were downregulated greater than 5.0-fold in disease (Table 1). The highest fold change observed in the upregulated gene set was a 38.2 fold induction of the pancreatitis-associated protein 1 (Pap 1) gene (Table A). In comparison, 11 genes were downregulated greater than 40-fold, with the greatest fold reduction represented by amylase 1 (Amy 1) at −126.9-fold (group 13, Table 2).

Using the gene annotation method outlined above, the 171 disease-related genes were categorized into groups based upon their cellular function. From this analysis, the disease-associated genes were clustered into 22 functional classifications. Table 2 lists, according to functional classifications, the 149 markers that were not known prior to the invention to be associated with IBD. The remaining 22 markers that were known prior to the invention to be associated with IBD are listed without functional classifications in Table A. Genes associated with antigen processing and presentation were upregulated in disease (group 1, Table 2). These genes include major histocompatibility complex (MHC) class I and class II molecules, MHC class II-associated invariant chain, proteosome subunits, and antigen transporter polypeptides (several of these are included in Table A). Increased expression of these genes as well as genes encoding T cell receptors (group 7, Table 2) support the role of an aberrant immunological response in the colonic disease of this model (Breban et al. (1996) J. Immunol. 156:794-803; Taurog et al. (1999) Immunol. Rev. 169:209-23). Taken together, the expression profile suggests that enhanced or aberrant antigen processing is associated with disease in this model and is consistent with observations for human IBD.

As expected, genes involved in an inflammatory response were also upregulated in the diseased colon (group 2, Table 2). These included genes encoding interferon regulatory factors, chemokines, complement proteins and immunoglobin (group 2, Table 2). The pancreatitis-associated proteins Pap1 and Pap3 were also upregulated in the diseased colon (Table A). These proteins were originally identified as markers of acute pancreatitis (Bodeker et al. (1998) Digestion 59:186-91) but have also been shown to be upregulated in the inflamed rat intestine (Sansonetti et al. (1995) Scand. J Gastroenterol. 30:664-69; Iovanna et al. (1993) Am. J. Physiol. 265:G611-18) and in the colon of patients suffering from ulcerative colitis and Crohn's disease (Lawrance et al., supra). Signal transduction and transcription factor proteins associated with an inflammatory response (Stat1 and NfKb1; groups 6 and 5, respectively, Table 2) were also upregulated in disease. Expression of genes encoding heat shock proteins was downregulated in disease (group 2, Table 2).

Genes associated with cell death or apoptosis were upregulated in diseased colonic tissue. This gene set includes caspase family members, Bak and granzyme b (group 8, Table 2). Also modulated in the HLA-B27 rat colonic diseased tissue are genes associated with the development and maintenance of cellular and structural components of the gastrointestinal mucosa. These included genes that are categorized under mesodermal development (group 4, Table 2), cell adhesion molecules (group 18, Table 2), cytoskeletal structural proteins (group 19, Table 2) and muscle filaments (group 20, Table 2). Genes contained within each of these groups were both up- and downregulated in disease, which perhaps may illustrate the reciprocal forces of damage and repair of the gastrointestinal mucosa in this model.

By far the most noteworthy changes in gene expression were seen in genes encoding metabolic enzymes. These metabolic genes are mostly downregulated in disease and are associated with lipid (group 11, Table 2), protein (group 14, Table 2), steroid (group 12, Table 2) and carbohydrate (group 13, Table 2) metabolism. All the genes categorized as being involved in the metabolism of proteins are members of the serine protease superfamily and associated with the hydrolysis of dietary protein. Similarly, all the genes contained in the steroid and lipid metabolism groups are involved in the metabolism of dietary steroids and fats. Only 3 genes out of the 33 genes associated with metabolism of dietary substrates and listed in Table 2 are upregulated in disease. Both Hk2 (hexokinase 2) and Pfkp (phosphofructokinase C) are in the carbohydrate metabolism group and are upregulated in disease (group 13, Table 2). Hk2 controls the entry of free glucose into the glycolytic pathway and Pfkp represents the commitment step of glucose into the glycolytic pathway. However Aldob (aldolase b), which is involved in the control of the sixth step of glycolysis, was downregulated in disease. Fabp-5 (cutaneous fatty acid-binding protein) is in the lipid metabolism group and is upregulated in disease (group 11, Table 2). Fabp-5 is thought to play an important role in the transport and metabolism of fatty acids in epidermis (Watanabe et al. (1997) J. Dermatol. Sci. 16:17-22; Watanabe et al. (1996) Arch. Dermatol. Res. 288:481-483). However there are no previous reports of its expression in the colon. Pla2g2a (platelet phospholipase A2, see Table A) is also involved in lipid metabolism and is also upregulated; due to its role in the eicosanoid biosynthesis pathway, it would be expected to be upregulated in inflammatory tissue.

All the proteins encoded by the metabolic enzymes noted above are secreted enzymes that must be packaged into secretory vesicles prior to export from the cell. Analysis of diseased tissue showed a decrease in the expression of genes encoding integral membrane proteins of secretory [Sip9 (An et al. (2000) J. Biol. Chem. 275:11306-11; Edwardson et al. (1997) Cell 90:325-33); and Gp2 (Rindler et al. (1990) Eur. J. Cell Biol. 53:154-63; Hoops et al. (1993) J. Biol. Chem. 268:25694-705)] and endocytic vesicles [Stx7 (Mullock et al. (2000) Mol. Biol. Cell 11:3137-53)] (group 10, Table 2). A significant decrease in the expression of genes encoding regulators of intracellular membrane trafficking was also detected [Mss4 (Burton et al. (1993) Nature 6411:464-67; Burton et al. (1994) EMBO J. 13:5547-48); and Pyy (Fujimiya (2000) Peptides 21:1565-82)] (group 10, Table 2). Therefore not only is there a defect in the expression of lipases and proteases in the colonic tissue of the HLA-B27 rat, but there is also a corollary decrease in genes encoding proteins involved in the control of vesicle trafficking and in structural components of exocytic and endocytic vesicle membranes.

### Example 1.12

### Results: rhIL-11 Ameliorates Signs of Inflammatory Bowel Disease

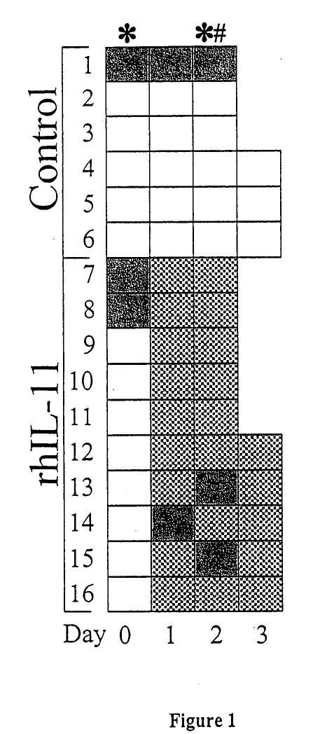

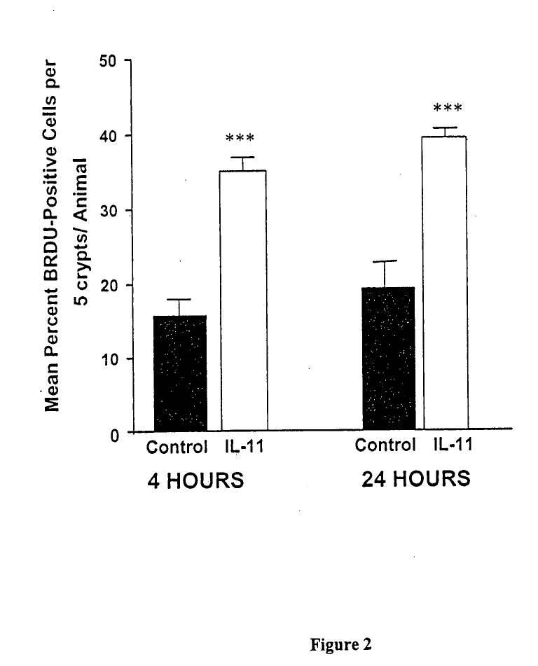

The HLA-B27 rat develops inflammatory bowel disease that is clinically manifested as diarrhea and lesions in intestinal tissues. HLA-B27 rats receiving 2 doses of rhIL-11 (37.5 μg/kg), 48 hrs apart and killed 4 hr after the last dose, had improved stool character, exhibited by increased number of days of normal stool character relative to number of days of diarrhea (Peterson et al. (1998) supra). FIG. 1 shows the reduced incidence of days with diarrhea and loose stool in the rhIL-1′-treated HLA-B27 rats compared to vehicle-treated HLA-B27 rats. The majority of the rhIL-11-treated animals show a change from diarrhea to normal stool as early as the first day (24 hrs) after receiving rhIL-11 treatment (compare animals 1-6 with animals 7-16, FIG. 1). Only three rhIL-1-treated animals (animals 13-15), failed to continue having persistent days of normal stool character. However, each rhIL-11-treated animal exhibited normal stool character on the day of sacrifice (Day 2, animals 7-11; Day 3, animals 12-16). In comparison, no vehicle-treated animal had normal stool character at any day during the study, and all animals except one consistently exhibited diarrhea (animals 1-6, FIG. 1). Histological analysis was performed on tissue isolated at both the Day 2 and Day 3 time points. rhIL-11-treatment significantly reduced the total lesion score compared to vehicle-treated rats at both time points (Table 3). rhIL-11 treatment also significantly reduced the levels of IFN-γ, IL-1β and TNFα mRNA in the colon of the HLA-B27 rats as measured by TaqMan™ RT-PCR.

### Example 1.13

### Results: rhIL-11 Affects Inflammatory Bowel Disease-Related Gene Expression

rhIL-11 treatment significantly modulated the expression of 35 genes in the colonic tissue of HLA-B27 rats compared to vehicle-treated rats. Twenty-seven of these genes were identified as disease-associated (Table 4 shows 26 of these; the remaining gene, Rib1, is listed in Table A). Sixteen of these disease-associated genes are members of the lipid and protein metabolizing groups, and 15 of these were significantly upregulated by rhIL-11 treatment (members of groups 11 and 14, Table 4). Also increased upon rhIL-11 treatment was mRNA encoding pancreatic secretory trypsin inhibitor (Spink2), an inhibitor of serine proteases. The parallel upregulation of Spink2 in conjunction with serine proteases, such as the trypsinogens, is thought to function to ensure protection against premature activation of proteolysis (Graf et al. (2000) Pancreas 21:181-90). Pancreatic amylase (Amy1) was also upregulated by rhIL-11-treatment (Table 4). rhIL-11 treatment returned to normal levels genes encoding proteins involved with the metabolism of protein, lipids and oligosaccharides in the colon, as well as several other genes (compare “Fold Δ A” column in Table 4 with “Fold Δ” column in Table 2; see also “Fold Δ B” column in Table 4, which shows the relative fold-change in the nondiseased Fischer 344 rat colon compared to the vehicle-treated HLA-B27 rat colon). Pancreatic ribonuclease (Rib1) was also upregulated in response to rhIL-11 treatment (by a factor of 53.50). Rib1 was downregulated in the disease state (by a factor of −50.65, Table A). This protein catalyzes the endonucleolytic cleavage of 3′-phosphomononucleotides and 3′-phosphooligonucleotides for the digestion of RNA and therefore shares the same functionality as the other metabolic genes upregulated by rhIL-11.

Two of the disease-related genes that were upregulated by rhIL-11 in the colon of the HLA-B27 rats encode proteins localized in the membranes of secretory vesicles [Sip9 and GP-2, Table 4; (An et al. (2000) J. Biol. Chem. 275:11306-11; Rindleretal. (1990) Eur. J. Cell. Biol. 53:154-63)]. The remaining genes upregulated by rhIL-11 treatment are members of the membrane transporter category (Aqp3; group 22, Table 2) and cation channel proteins (plasmolipin; group 21, Table 2). Aqp3 is a member of the aquaporin (Aqp) water channel protein family and is the prototype member of the Aqp proteins that transport glycerol and urea in addition to water (Ishibashi et al. (1994) Proc. Natl. Acad. Sci. USA 91:6269-73). Plasmolipin is a tetraspan protein that is highly expressed by myelinating glial cells and is associated with CNS and PNS myelin (Gillen et al. (1996) Eur. J. Neurosci. 405-14; Fischer et al. (1994) Neurochem. Res. 19:959-66).

Expression levels of five disease-related genes upregulated in the diseased colon were decreased by rhIL-11-treatment (Table 4). These genes include high mobility group protein I (Y) (Hmgiy), 1-kappa B alpha chain (Nfkβ1α), Hk2 and Fabp5 (both discussed above) and rat MHC class II-like beta chain (RT1.DMβ). These genes were downregulated by rhIL-11 in the 2.5 to 4.05 range. The Hmgiy gene is a member of a three gene family group of high-mobility group (HMG) mammalian nonhistone nuclear proteins and is thought to participate in numerous biological processes (e.g., transcription, replication, retroviral integration, genetic recombination) by its ability to recognize and alter the structure of both DNA and chromatin substrates (Reeves et al. (2000) Environ. Health. Perspect. 108:803-09). I-kappa B alpha chain (Nfkβ1α) is an inhibitor of the transcriptional factor NF-κB, and is rapidly induced following adherence of murine and human monocytes (Haskill et al. (1991) Cell 65:1281-89). RT1.DMβ is a MHC class II associated molecule that is the rat ortholog to the human leukocyte antigen HLA-DMβ (Hermel and Monaco (1995) Immunogenetics 42:446-47 (published erratum appears in (1996) Immunogenetics 44:487)). The HLA-DM gene has been shown to function in the synthesis of MHC class II receptors by catalyzing the removal of an invariant chain derived peptide (CLIP) from newly synthesized class II molecules to free the peptide binding site for acquisition of antigenic peptides (Weber et al. (1996) Science 274:618-21).

### Example 1.14

### Results: Identification of rhIL-11-Respondent Nondisease-Related Gene Expression

rhIL-11-treatment also modulated the expression of eight genes that were not identified as disease related (not found to be significantly different in the Fischer 344 and vehicle-treated HLA-B27 comparison); four of the genes were upregulated, and four were downregulated. Six of those genes are listed as genes of the invention in Table 5; the two other genes, RegI and TFF2, both of which were upregulated, were known to be associated with IBD prior to the invention.

Four of the genes were upregulated by rhIL-11-treatment and were found to encode known or putative growth factors of intestinal epithelial cells. Neither GeneChip® or TaqMan™ analysis detected any of these four genes in colons of vehicle-treated HLA-B27 rats. The most highly induced genes encode two members of the Reg gene superfamily (Okamoto et al. (1999) J Hepatobiliary Pacreat. Surg. 6:254-62), the Regeneration I (RegI) and Regeneration III (RegIII) proteins, which were induced by rhIL-11 treatment greater than 30 fold. These proteins have been implicated to play an important role in the regeneration of cells and tissues of the gastrointestinal tract and pancreas ((Okamoto et al. (1999) J Hepatobiliary Pacreat. Surg. 6:254-62; Asahara et al. (1996) Gastroenterol. 111:45-55; Kawanami et al. (1997) J. Gastroenterol. 32:12-18; Kazumori et al. (2000) Gastroenterol. 119:1610-22; Kobayashi et al. (2000) J. Biol. Chem. 275:10723-26; Perfetti et al. (1996) J. Mol. Endocrinol. 17:79-88; Zenilman et al. (1997) Ann. Surg. 225:327-32). RegI (accession no. M62930) expression was induced 65.92-fold by rhIL-11 treatment. RegIII expression was induced 31-fold by rhIL-11 (Table 5). Spasmolytic polypeptide (TFF2) and Insulin II (Ins2) were also induced in the HLA-B27 rat in response to rhIL-11 treatment, however at a much lower frequency. TFF2 (accession no. M97255) expression was induced 3.21-fold by rhIL-1-treatment. TFF2 is a member of the trefoil peptide family, which has been shown to participate in the protection and repair of gastric mucosa (Playford et al. (1997) J. R. Coll. Phys. Lond. 31:37-41). Insulin II (Ins2) showed the lowest-fold induction in response to rhIL-11 in this group (2.52-fold, Table 5). Ins2 is one of two nonallelic insulin genes that have been found in the rat genome (Giddings et al. (1988) J. Biol. Chem. 263:3845-49). It is a nonpancreatic source of insulin in some adult (Devaskar et al. (1993) Regul. Pept. 48:55-63) and embryonic organs (Giddings et al. (1988) J. Biol. Chem. 263:3845-49; Giddings et al. (1989) J. Biol. Chem. 264:9462-69; Giddings et al. (1990) Mol. Endocrinol. 4:1363-69) prior to the formation of the pancreas. This is the first report showing its expression in the adult rat intestine; however, it was only present in rhIL-11-treated HLA-B27 rats.

The RegI gene has previously been identified as a marker of IBD (Lawrance et al., supra). However, here RegI is identified as indicative of the healing process. Upon treatment with rhIL-11, RegI expression increased by a factor of 65.92 in the HLA-B27 rat, thus supporting the hypothesis that RegI expression is beneficial in promoting healing. RegIII, Ins2 and TFF2 are also identified as indicators of healing, and their expression, individually or in combination, along with RegI, may be beneficial in promoting healing in IBD. TFF2 has been hypothesized to be involved in healing in some forms of IBD (Thim et al., International Pat. Appln. Publication No. WO 02/46226). As stated in Table 5 for RegIII and Ins2, the genes RegI and TFF2 were called “absent” in the vehicle-treated rat, therefore the fold-change values for these genes are much larger than described.

Expression levels of four genes were reduced by rhIL-11-treatment in the rhIL-11 respondent gene list (Table 5). Junction plakoglobin (Jup) is a member of the beta-catenin family of cell adhesion molecules and is a common junction plaque protein of the intercellular adhesive junctions. Jup acts to anchor intermediate filaments at membrane-associated plaques in adjoining cells by linking them to the actin cytoskeleton (Zhurinsky et al. (2000) J Cell Sci. 113:3127-39). It also participates in adhesion-mediated signaling by binding and activating transcription factors mediating Wnt signal transduction (id.). The ps20 protein is a member of a family of small secreted serine protease inhibitors called the whey acid protein (WAP) four-disulfide core domain proteins (Larsen et al. (1998) J. Biol. Chem. 273:4574-78). This family of proteins exhibits a fundamental role in growth control, cellular differentiation and tissue remodeling. Recombinant ps20 protein has growth-inhibition effects on epithelial-derived cells in vitro (id.; Rowley et al. (1995) J. Biol. Chem. 270:22058-65). The VL30 element is a retrotransposable element that has become incorporated into the rat genome from a retroviral insertion (French et al. (1997) Biochim. Biophys. Acta 1352:33-47). It has been shown to be a possible prototype of growth-regulated genes and has been isolated in many subtractive cDNA libraries constructed to isolated growth-associated genes (id.). rhIL-11-treatment reduced the expression of the VL30 element 2.96-fold. Glutathione synthetase catalyzes the synthesis of glutathione, which is thought to act as a cellular redox buffering agent.

### Example 1.15

### Results: Localization of Intestinal Epithelial Growth Factor Expression In Vivo

In situ hybridization analysis was performed on colonic tissues isolated from rhIL-1- and vehicle-treated HLA-B27 rats to localize the expression pattern of the RegI, RegIII, TFF2 and Ins2 genes. Signal for the presence of each gene was seen in the colonic biopsies isolated from rhIL-11-treated HLA-B27 rats. There was no signal detected in colonic tissue isolated from vehicle-treated HLA-B27 rats. These results support both the GeneChip® and RT-PCR results showing the absence of message for each of the four intestinal epithelial growth factors in the colon of vehicle-treated HLA-B27 rats. The expression pattern of all four genes in the colon of rhIL-11-treated HLA-B27 rats was essentially the same. The expression of each gene was localized to the cytoplasm of epithelial cells. Expression was seen in epithelial cells ranging from the bottom of the crypt to the luminal surface in longitudinal-oriented colon sections and in all the epithelial cells in the circumference of a cross section through the crypts.

### Example 1.16

### Results: rhIL-11 Induced Proliferation of Intestinal Epithelial Cells In Vivo

To investigate the effect of rhIL-11 treatment on the proliferation of intestinal epithelial cells in vivo, 500 μg/kg bromodeoxyuridine (BrdU) was administered by intraperitoneal injection at the time of the second dose of rhIL-11 treatment (Day 2). BrdU is a uridine analog and is incorporated in the DNA of cells undergoing cellular replication. Animals were sacrificed 4 and 24 hr following administration of BrdU and the localization and enumeration of BrdU-positive cells was analyzed using immunohistochemical techniques with an anti-BrdU antibody. The number of BrdU-positive cells in the colons was calculated for each animal in each treatment group, averaged and subjected to statistical analysis. At both the Day 2 and Day 3 sacrificial time points, there were significantly more BrdU-positive epithelial cells in the rhIL-11-treated animal compared to the animals treated with vehicle, indicating that the administration of rhIL-11 caused a trophic response in the HLA-B27 rat colon and expanded the proliferative compartment of the intestinal epithelial cells by approximately 2-fold (FIG. 2).

### Example 1.17

### Results: rhIL-11 Treatment of Fischer 344 Rats had no Significant Effect

Comparison of colonic RNA isolated from Fischer 344 rats treated with or without 37.5 μg/kg rhIL-11 did not show significant gene expression differences in the levels of any of the genes listed in Table 5, or RegI and TFF2. In addition, there was no effect histologically or in the incorporation rate of BrdU in colonic intestinal epithelial cells between the vehicle- and rhIL-11-treated Fischer 344 rats.

### Example 1.18

### Discussion

It previously has been shown that rhIL-11 decreases the histological and clinical signs of IBD in the HLA-B27 rat and its activity is associated with the downregulation of inflammatory cytokine expression and reduction of myeloperoxidase activity in intestinal tissue (Peterson et al. (1998) supra). The present invention extends analysis of the molecular effects of rhIL-11 in this model by global expression analysis. The use of global expression analysis has allowed identification of previously unrecognized pathways in disease and rhIL-11-related mechanisms in this rat model of IBD. The Fischer 344 rat is the background strain for the transgenic HLA-B27 rat and differs from the HLA-B27 rat only in the absence of the human HLA-B27 and β2-microglobulin gene expression (Hammer et al. (1990) Cell 63:1099-12). Therefore gene expression differences between the Fischer 344 and HLA-B27 rat strains have been defined as disease-related. This comparison allowed identification of a gene set differentially expressed in the diseased colon associated with IBD in the HLA-B27 rat.

Chronic inflammatory bowel disease in humans has been shown to be associated with increased class II MHC expression (Braegger (1994) Acta Paediatrica Suppl. 83:18-21). Studies have shown that the colonic epithelium of Crohn's disease patients develop strong expression of the HLA-DR antigen (Lawrance et al., supra); Selby et al. (1983) Clin. Exp. Immunol. 53(3):614-18; Hirv et al. (1999) Scand. J Gastroenterol. 34:1025-32). This study shows that the rat ortholog of the human HLA-DRB1 allele (RT1-Dα1 and RT-Dβ1) was significantly increased in the inflamed colon of the HLA-B27 rat. In addition, rat MHC class II RT1-Bβ and RT1-Bα alleles (orthologs of human HLA-DQB1 and HLA-DQA1, respectively) were also elevated in the HLA-B27 rat. Studies have also indicated that the human ortholog of the rat RT-1B is associated with the genetic susceptibility to IBD in humans (Annese et al. (1999) Eur. J. Hum. Genet. 7:567-73; Satsangi et al. (1996) Lancet 347:1212-17; Mayer et al. (1991) Gastroenterol. 100:3-12). Therefore, this study has correlated the expression of these class II MHC alleles with both human IBD and this rat model, suggesting a related disease pathway.

T cells recognize processed antigen in association with MHC molecules. Antigen processing involves multicatalytic proteinase complexes called proteosomes (Roitt et al. ((1998) in Immunology (4^{th} ed.) Cook, ed., Barcelona, Spain: Mosby:7.11). Processed peptides are transported into the rough endoplasmic reticulum (RER) by ABC transmembrane transporters (Joly (1998) Immunol. Today 19:580-85; Abele et al. (1999) Biochim. Biophys. Acta 1461:405-19). The expression of 4 genes encoding individual proteosome subunits (Psrnb2, 4, 8 and 9) were significantly upregulated in the colons of HLA-B27 rat compared to the nondiseased Fischer 344 colon. There was also a concomitant upregulation of the genes encoding the ABC transmembrane transporter molecules Abcb3 and Abcb1 in the HLA-B27 rat colon (Table 2). Therefore, expression levels of multiple genes associated with antigen processing have been identified as upregulated in the HLA-B27 colon.

MHC class I and II molecules are synthesized and assembled in the RER (Roitt et al. (supra)). Class II α and β chains are found in the RER associated with a polypeptide derived from MHC-class II associated invariant chain (Ii) (Alfonso et al. (2000) Ann. Rev. Immunol. 18:113-42). The MHC class II α,β Ii complex is transported through the Golgi complex to an acidic endosomal or lysosomal compartment, where a remnant of the Ii peptide (CLIP) is removed from the MHC complex in order to expose the antigen binding site. The removal of the CLIP peptide is catalyzed by the MHC class II-like protein HLA-DM (id.). Expression of genes encoding the rat invariant chain (Cd74) and the rat ortholog of the human α and β chains of HLA-DM (RT1.DMα and RT1.DMβ) are upregulated in the HLA-B27 rat. Consequently, increased expression of genes involved in antigen processing and assembly of MHC class II molecules in the inflamed colon of HLA-B27 rats have been identified.

rhIL-11 treatment of HLA-B27 rats resulted in levels of mRNA encoding the β chain of the RT1-DM reduced 4.05-fold compared to the vehicle treated HLA-B27 rats (RT1-DMβ, Table 4). This was the only gene involved in antigen presentation or processing that was affected by rhIL-11-treatment. Antigen presenting cells deficient in HLA-DMβ chain expression (Mellins et al. (1990) Nature 343:71-74; Morris et al. (1994) Nature 368:551-54) are defective in presenting antigen to T cells due to CLIP peptide occupation of the antigen binding site of Class II MHC proteins (Weber et al. (1996) Science 274:618-21). Thus, a 4.05-fold reduction of this key gene in the rhIL-11-treated HLA-B27 rat may be sufficient to inhibit antigen presentation in vivo, affecting a key step in the antigen presentation pathway and modulating antigen presentation in the colon. Therefore, a possible mechanism for disease amelioration by rhIL-11 may be a reduction in antigen presentation leading to a reduced T cell response in the colon.

By far the most striking differential expression associated with disease was the reduced expression of gene sets involved with the metabolism of proteins and lipids. The widespread downregulation of these genes in the inflamed colon is indicative of a major disruption in metabolism and energy utilization in IBD. A similar decrease in the expression of genes associated with the metabolism of protein, lipid and carbohydrate has also been reported in colonic tissue isolated from ulcerative colitis (UC) patients (Lawrance et al., supra). Roediger initially suggested that the colonic epithelium of UC was an energy-deficient tissue based upon in vitro studies of colonocytes isolated from UC patients (Roediger (1980) Lancet 2(8197):712-15). Roediger reported that the oxidization of a fatty acid (n-buterate) was reduced in both quiescent and active UC, and the level of reduction correlated with the state of disease. However, the cells were not completely energy deficient as enhanced glucose oxidation occurred in these cells, perhaps to compensate for the defect in the oxidation of fatty acids. The present study shows evidence of a similar phenomenon occurring in the colons of HLA-B27 rats. Reductions in the expression of genes encoding proteins associated with phospholipid metabolism (Pnlip, Cel, Pnliprp2, Pla2g1b, Clps, Scd2) and fatty acid β oxidation (Hmgcs2, Ratacoa1 and Cytb), coupled with a significant increase in the expression of two genes (Hk2 and Pfkp) encoding major proteins controlling glucose metabolism, were observed.

Treatment of HLA-B27 rats with rhIL-11 to ameliorate disease restored the expression levels of many genes encoding metabolic proteins to the expression level seen in a nondiseased colon. Thus, the restoration of normal metabolic processes is associated with the amelioration of disease with rhIL-11 treatment. One feature of many of these metabolic genes upregulated by rhIL-11 treatment is that they encode secreted proteins that must be processed and packaged into secretory vesicles for export from the cell. Two genes that were upregulated by rhIL-11-treatment of the HLA-B27 rat have been shown to be integral-membrane proteins specifically incorporated into the membranes of secretory vesicles. Syncollin (Sip9) and the Zymogen granule membrane protein (Gp2) are both described as integral membrane proteins of pancreatic zymogen granules, the secretory vesicles of the pancreas (An et al. (2000) J. Biol. Chem. 275:11306-11; Rindler et al. (1990) Eur. J. Cell. Biol. 53:154-63). Sip9 is upregulated 104.92-fold, and Gp2 19.5-fold, by rhIL-11-treatment in HLA-B27 rat colons compared to vehicle treated HLA-B27 rats (Table 4). Sip9 expression has also previously been detected in rat colon, and additionally in the spleen and duodenum (Tan et al. (2000) Am. J. Physiol Gastrointest. Liver Physiol. 278:G308-20). Sip9 possibly regulates the control of secretory vesicle translocation in a Ca²⁺-mediated process (Edwardson et al. (1997) Cell 90:325-33). Its expression in the duodenum is increased in response to feeding, suggesting a role for syncollin in the secretion of digestive enzymes (Tan et al. (2000) Am. J. Physiol. Gastrointest. Liver Physiol. 278:G308-20). Gp2 is the major protein of the pancreatic zymogen granule membrane and is localized to the apical membrane of pancreatic acinar cells (Rindler et al. (1990) Eur. J. Cell Biol. 53:154-63). Similar in vitro experiments have shown that both Sip9 and Gp2 localize to the membrane of secretory granules containing Amy2 in AtT20 cells (Hoops et al. (1993) J. Biol. Chem. 268:25694-505; Hodel et al. (2000) Biochem. J. 350:637-43). Therefore, rhIL-11 restores the levels of mRNA encoding metabolic proteins that are exported from the cell in secretory vesicles, and proteins that localize in the membrane of secretory vesicles. This result implies that rhIL-11 restores the exocytotic process of epithelial cells, which is indicative of a healing or restorative response.

Various in vivo studies have indicated that rhIL-11 treatment promotes the growth of epithelial cells. Orazi et al. ((1996) Lab. Invest. 75:3342) reported that rhIL-11 treatment of mice after cytoablative therapy with 5-FU and radiation resulted in rapid intestinal epithelium recovery mediated by increased mitotic activity of crypt cells. In rat models of short bowel resection surgery, rhIL-11 enhanced crypt cell mitotic rates and increased mucosal mass (Fiore et al. (1998) J Pediatr. Surg. 33:24-29; Alavi et al. (2000) J. Pediatr. Surg. 35:371-74; Liu et al. (1996) J. Pediatr. Surg. 31:1047-51). In the present study, rhIL-11 treatment increased the BrdU-labeling index in colonic epithelial cells of HLA-B27 rats. This supports the role of rhIL-11 as a mediator of epithelial growth.

rhIL-11 treatment of HLA-B27 rats results in the upregulation of expression of four genes that may mediate the proliferative effects. Two genes are members of the Reg gene superfamily (Okamoto et al. (1999) J. Hepatobiliary Pancreat. Surg. 6:254-62) that were originally identified as potential growth factors for pancreatic islet cells (Terazano et al. (1988) J. Biol. Chem. 263:2111-14). However, Reg I expression has also been detected in organs other than the pancreas, including normal gastrointestinal mucosa (Kawanami et al. (1997) J Gastroenterol. 32:12-18). RegI gene expression is reported to increase during the healing of damaged gastric mucosa, specifically in enterochromaffin-like (ECL) cells (Kazumori et al. (2000) Gastroenterol. 119:1610-22). Gastrin has long been known as a trophic factor of gastric mucosa (Johnson et al. (1993) in Gastrin, Walsh, ed. Raven Press, New York, pg. 285-300). Gastrin stimulates the production of RegI protein in ECL cells, linking the expression of RegI protein to the ability of gastrin to induce proliferation of mucosal cells (Fukui et al. (1998) Gastroenterol. 115:1483-93). This result supports the role of RegI gene in healing of gastrointestinal mucosal lesions (Chiba et al. (2000) J. Gastroenterol. 35:52-56). RegIII is also a member of the Reg gene family (Okamoto (1999) J. Hepatobiliary Pacreat. Surg. 6:254-62). Members of the RegIII subclass have been shown to be expressed in normal Paneth cells of the human gastrointestinal tract (Christa et al. (1996) Am. J. Physiol. 271:G993-1002).

TFF2 is a member of the trefoil family peptides, which are major secretory products of mucus cells of the gastrointestinal tract that show increased expression at the sites of mucosal injury (Playford et al. (1997) J. R. Coll. Phys. London 31:37-41; Murphy (1998) Nutrition 14:771-74). TFF2 is expressed within 30 minutes following mucosal damage (Alison et al. (1995) J. Pathol. 175:405-14) and has been shown to stimulate cell migration in vitro ((Playford et al. (1997) J. R. Coll. Phys. London 31:37-41). One of the earliest processes following mucosal injury is a rapid migration of cells from the margins of the damaged region over the denuded area to reestablish epithelial integrity (id.). These results suggest that TFF2 is an important mediator of the migration of epithelial cells to heal intestinal lesions. Orally administered recombinant TFF2 has been effective in treating aspirin-induced gastric injury when administered before or concomitantly with aspirin (Cook et al. (1998) J. Gastroenterol. Hepatol. 13:363-70). Tran et al. ((1999) Gut 44:636-42) found that TFF2 is negligibly expressed in the normal colon but endogenous concentrations of TFF2 protein increased following dinitrobenzene sulfonic acid-induced injury. Orally administered rhTFF2 in this model accelerated healing and reduced the levels of myeloperoxidase activity in the colon. The induction of TFF2 expression by rhIL-11 treatment in the HLA-B27 rat may contribute to the observed reduction of myeloperoxidase activity, as well as enhanced lesion healing, seen previously (Peterson et al. (1998) supra). The role of these several growth factors and putative growth factors (i.e., RegI, RegIII, Ins2 and TFF2) in epithelial growth and restoration and their induction during amelioration of disease suggests a therapeutic use to induce healing of the lesions associated with IBD.

No increased expression of RegI, RegIII, TFF2 or Ins2 was detected in the colon of rhIL-11-treated Fischer 344 rats. Treatment of normal Fischer 344 animals with rhIL-11 also had no effects on BrdU incorporation rate in intestinal epithelial cells of Fischer 344 rats in vivo, indicating that a disease or damaged state must be present for rhIL-11 activity. rhIL-11, therefore, may be inducing these epithelial growth factors in synergy with factors present in the damaged, but not normal, intestine. Expression of these epithelial growth factors can be viewed as evidence of a reparative process in gastrointestinal tissue, as opposed to as a marker of disease.

TABLE 1
Numerical distribution of differentially regulated genes
in colon of HLA-B27 rats*

| Upregulated | | Downregulated | |
|---|---|---|---|
| Fold-Change | No. of Genes | Fold-Change | No. of Genes |
| 2.4 to 5.0 | 77 (86.5%) | −2.4 to −5.0 | 46 (56.1%) |
| >5.0 to 10.0 | 6 (6.7%) | −5.0 to −10.0 | 12 (14.6%) |
| 10.0 to 20.0 | 5 (5.6%) | −10.0 to −20.0 | 5 (6.1%) |
| 20.0 to 40.0 | 1 (1.1%) | −20.0 to −40.0 | 8 (9.8%) |
| 40.0 to 60.0 | — | −40.0 to −60.0 | 3 (3.7%) |
| 60.0 to 100.0 | — | −60.0 to −100.0 | 6 (7.3%) |
| 100.0 to 130.0 | — | −100.0 to −130.0 | 2 (2.4%) |

NY_MAIN 313727v1
*Upregulated indicates genes that are overexpressed compared to Fischer 344 control rats (i.e., >2.4-fold); downregulated indicates genes that are underexpressed using the same comparison. For each range of fold-change, percentages of the total number of genes in each category are given (total upregulated = 89; total downregulated = 82).

TABLE 2
Disease related genes in the HLA-B27 rat colon

| | | | |
|---|---|---|---|
| Symbol | Accession | Fold Δ | P value |

(1) Antigen Processing and Presentation

| | | | |
|---|---|---|---|
| Abcb1 | X57523 | 13.141 | 0.000114 |
| Psmb9 | D10757 | 9.0157 | 5.63E−05 |
| RT1.DMb | U31599 | 8.8647 | 4.27E−07 |
| Psmb8 | D10729 | 5.9012 | 2.93E−08 |
| RT1-S3 | AI235890 | 3.6559 | 7.29E−05 |
| Psme2 | D45250 | 3.4811 | 2.32E−06 |
| Abcb3 | X63854 | 3.1633 | 4.1E−05 |
| Psmb4 | L17127 | 2.4336 | 0.000415 |

(2) Inflammatory Response

| | | | |
|---|---|---|---|
| Scya5 | AI009658 | 14.902 | 0.002242 |
| Mcpt8 | U67911 | 10.684 | 4.58E−06 |
| Cx3c | AF030358 | 8.1481 | 0.009426 |
| Irf7 | AA799861 | 5.3623 | 0.012468 |
| Mcpt2 | J02712 | 4.9686 | 1.69E−05 |
| Irf1 | M34253 | 4.3386 | 6.05E−05 |
| C4 | U42719 | 4.2836 | 1.42E−05 |
| Mcpt1 | AF063851 | 3.75 | 0.000729 |
| Lyz | L12459 | 3.6154 | 0.001233 |
| Fcgr3 | M32062 | 3.3333 | 0.03347 |
| Daf | AF039583 | 2.9333 | 0.001748 |
| Scya2 | X17053 | 2.7778 | 0.000679 |
| Mcpt10 | U67913 | 2.6852 | 0.002203 |
| Aif1 | U17919 | 2.6667 | 8.37E−05 |
| Mcpt4 | U67907 | 2.6471 | 0.015179 |
| L07402 | L07402 | 2.483 | 0.038317 |
| Hspf1 | AA859648 | 2.4257 | 0.000316 |
| Mep1a | S43408 | −2.7429 | 0.002335 |
| Serpinh1 | M69246 | −3.0462 | 0.000324 |
| Hsj4 | AA848268 | −3.0783 | 0.000533 |
| II18 | U77777 | −3.1385 | 0.000615 |
| D29960 | D29960 | −3.3429 | 0.019374 |
| Hsp25 | M86389 | −8.4 | 0.000547 |

(3) Cell Growth and Maintenance

| | | | |
|---|---|---|---|
| Gstm5 | J03752 | 4.4662 | 0.031668 |
| Gpx2 | AA800587 | 4.4215 | 0.001191 |
| Alpl | J03572 | 2.9106 | 0.000861 |
| UNK_AI231007 | AI231007 | 2.8736 | 1.1E−05 |
| UNK_M15114 | M15114 | 2.795 | 0.000493 |
| Ccnb1 | X64589 | 2.7564 | 0.041337 |
| Mcmd6 | U17565 | 2.4769 | 0.000199 |
| Cyp2d9 | J02869 | −2.6095 | 0.002515 |
| Gas6 | D42148 | −4.74 | 0.029528 |
| Gstm5 | U86635 | −4.9 | 0.000341 |

(4) Mesoderm Development

| | | | |
|---|---|---|---|
| Jag1 | L38483 | 2.6111 | 0.01576 |
| Retl2 | AF003825 | 2.4444 | 0.002999 |
| Dcn | X59859 | −2.7385 | 0.050477 |

(5) Transcription Factors

| | | | |
|---|---|---|---|
| Id2 | AI230256 | 3.4699 | 0.002435 |
| Gtf2f2 | L01267 | 3.0556 | 0.000154 |
| Nfkb1 | L26267 | 2.7679 | 3.45E−05 |
| Hmgiy | X62875 | 2.4667 | 0.001645 |
| Bteb1 | D12769 | −3.0889 | 0.004126 |

(6) Signal Transduction

| | | | |
|---|---|---|---|
| Stat1 | AA892553 | 19.167 | 6.52E−10 |
| Map3k12 | D49785 | 3.2552 | 0.004779 |
| Coro1a | AA892506 | 2.8901 | 0.005906 |
| Nrgn | L09119 | −3.2182 | 0.041229 |
| Sgk | L01624 | −8.304 | 9.47E−05 |
| Nfkbla | X63594 | 2.4667 | 0.006287 |
| Mir16 | AA891916 | −3.075 | 0.001518 |
| Guca2a | M95493 | −3.1526 | 0.034425 |
| Ralb | L19699 | −4.8 | 0.014861 |

(7) T Cell Receptors

| | | | |
|---|---|---|---|
| U76836 | U76836 | 3.9583 | 0.004172 |
| M18853 | M18853 | 3.5897 | 0.002442 |
| Cd3d | X53430 | 3.5294 | 0.010242 |
| Cd3g | S79711 | 2.619 | 3.97E−05 |

(8) Cell Death

| | | | |
|---|---|---|---|
| Casp1 | U14647 | 3.1564 | 9.72E−05 |
| Casp7 | AF072124 | 3.1034 | 0.01708 |
| Bak | H31839 | 2.6446 | 0.000414 |
| Gzmb | X66693 | 3.2353 | 7.75E−05 |

(9) Nucleic Acid Metabolism

| | | | |
|---|---|---|---|
| Xdh | AI172247 | 4.1844 | 0.00294 |
| Dnase1I3 | U75689 | 2.9825 | 9.03E−05 |
| Atic | D89514 | 2.8495 | 6.58E−05 |
| Pde4b | AA799729 | 2.4286 | 0.000866 |

(10) Secretory Vesicle Membrane Proteins

| | | | |
|---|---|---|---|
| Stx7 | AF031430 | −2.4188 | 0.0046 |
| Mss4 | X70496 | −3.0889 | 0.024642 |
| Pyy | M17523 | −4.1571 | 0.026496 |
| Gp2 | M58716 | −6.9 | 0.042991 |
| Sip9 | AF012887 | −60.4 | 0.040837 |

(11) Lipid Metabolism

| | | | |
|---|---|---|---|
| Cytb | AA875531 | −2.4632 | 0.021206 |
| Ratacoa1 | J02752 | −2.5875 | 0.003908 |
| Scd2 | U67995 | −2.9252 | 0.008446 |
| Fabp-5 | S69874 | 4.0566 | 2.42E−05 |
| Pnliprp2 | L09216 | −8.55 | 0.033168 |
| Ech1 | U08976 | −11.733 | 0.001213 |
| Clps | M58370 | −23.443 | 0.044435 |
| Hmgcs2 | M33648 | −31.3 | 0.002956 |
| Cel | X16054 | −38.367 | 0.03522 |
| Pnlip | D88534 | −70.4 | 0.033258 |
| Pla2g1b | D00036 | −91.25 | 0.047356 |

(12) Steroid Metabolism

| | | | |
|---|---|---|---|
| Apoa1 | J02597 | −2.8 | 0.039155 |
| Cyp3a13 | U46118 | −4.7714 | 0.011192 |

(13) Carbohydrate Metabolism

| | | | |
|---|---|---|---|
| HK2 | S56464 | 3.1159 | 7.97E−06 |
| Pfkp | L25387 | 2.7436 | 0.001942 |
| Ugt1a1 | S56937 | −2.7697 | 0.003486 |
| Hk1 | AI012593 | −2.939 | 0.032773 |
| Aldob | X02291 | −5.5667 | 0.040349 |
| Amy1 | V01225 | −126.9 | 0.038841 |

(14) Protein Metabolism

| | | | |
|---|---|---|---|
| Ivd | J05031 | −2.44 | 0.004632 |
| Pam | U52663 | −2.5556 | 0.022692 |
| Dpp4 | J04591 | −3.7 | 0.02497 |
| Dpep1 | AI170411 | −4.4 | 0.035976 |
| Cpa2 | M23721 | −13.994 | 0.035605 |
| Prss1 | J00778 | −21.643 | 0.044763 |
| Prss2 | V01274 | −31.2 | 0.045733 |
| Ela1 | L00117 | −32.1 | 0.038384 |
| Ctrc | S80379 | −42.05 | 0.038795 |
| Try3 | M16624 | −51.85 | 0.042792 |
| Cpa1 | J00713 | −60.563 | 0.04762 |
| Cpb | AI237825 | −77.3 | 0.044065 |
| Ela2 | L00124 | −89.28 | 0.032575 |
| Ctrb | K02298 | −122.75 | 0.043439 |

(15) Protease Inhibitors

| | | | |
|---|---|---|---|
| Spink2 | AA858673 | −5.7 | 0.03425 |
| Lxn | X76985 | 3.268 | 0.00752 |

(16) Carrier Protein

| | | | |
|---|---|---|---|
| Lck | AA800684 | 5 | 0.000179 |
| Ppicap | AF065438 | 4.0492 | 6.13E−05 |
| Ass | X12459 | 3.7963 | 0.000358 |
| Cyb5 | D13205 | −2.595 | 0.007214 |
| Sparcl1 | U27562 | −2.7743 | 0.050119 |
| Mt2 | M11794 | −5.8326 | 0.006495 |
| Mt1 | AI102562 | −6 | 0.019995 |
| Calb3 | K00994 | −27.456 | 0.002228 |

(17) Cell to Cell Communication

| | | | |
|---|---|---|---|
| Inha | M32754 | 3.0556 | 0.000271 |
| Ptprc | M10072 | 3.0357 | 0.028337 |
| Gjb2 | X51615 | 2.6235 | 0.002778 |
| Lamb2 | AI104225 | −2.7789 | 0.034713 |

(18) Cell Adhesion Molecules

| | | | |
|---|---|---|---|
| Glycam1 | L08100 | 3.125 | 0.026872 |
| U23056 | U23056 | −2.96 | 0.013641 |
| Itgb5 | S58644 | −3.8069 | 0.035651 |
| Thbs4 | X89963 | −14.1 | 0.029242 |

(19) Structural Proteins

| | | | |
|---|---|---|---|
| UNK_D13623 | D13623 | 2.8491 | 1.67E−07 |
| Serping1 | AA800318 | 2.5969 | 0.000571 |
| C1qb | X71127 | 2.5402 | 0.000405 |
| Timp2 | S72594 | −2.4125 | 0.029058 |
| Cola1 | M27207 | −2.4711 | 0.019656 |

(20) Muscle Filaments

| | | | |
|---|---|---|---|
| Tmpg | S82383 | 3.6198 | 0.001476 |
| Myh7 | X15939 | 2.5926 | 0.037018 |
| Agm | M64780 | 2.5641 | 0.007455 |
| Capg | AA894004 | 2.53 | 0.000157 |
| Tpm2 | L00382 | −2.4435 | 0.018809 |
| Myrl2 | S77900 | −2.7097 | 0.043541 |
| Myh11 | X16261 | −4.5158 | 0.033153 |

(21) Cation Channel Proteins

| | | | |
|---|---|---|---|
| UNK_AI639023 | AI639023 | 4.0617 | 0.001043 |
| Scnn1a | X70521 | −2.707 | 0.032002 |
| Z49858 | Z49858 | −7.4143 | 0.008124 |
| LOC64190 | L41254 | −9.4935 | 0.006045 |
| HKalpha2a | M90398 | −13.286 | 0.004767 |

(22) Membrane Transporters

| | | | |
|---|---|---|---|
| Ugtrel1 | D87991 | 2.6217 | 0.000257 |
| UNK_U87627 | U87627 | 2.4 | 0.006907 |
| SMVT | AF026554 | −2.6333 | 0.012244 |
| Slc16a1 | D63834 | −2.9333 | 0.032696 |
| Aqp8 | AB005547 | −3.9 | 0.042107 |
| Aqp3 | D17695 | −4.0714 | 0.025311 |

TABLE 3
Histological Lesion Scores in HLA-B27 Rats Treated with rhIL-11 or Vehicle***

| | Ulceration | Inflammation | Lesion depth | Fibrosis | |
|---|---|---|---|---|---|
| Group | (0-2) | (0-3) | (0-3) | (0-2) | Total score |
| Control Day 2 | 2.00 ± 0.00 | 2.33 ± 0.58** | 1.33 ± 0.58 | 1.33 ± 0.58 | 7.00 ± 1.73 |
| Control Day 3 | 1.33 ± 0.58* | 3.00 ± 0.00 | 1.00 ± 0.00 | 0.33 ± 0.58* | 5.67 ± 1.16 |
| rhIL-11 Day 2 | 0# | 1.20 ± 0.45# | 0# | 0* | 1.20 ± 0.45# |
| rhIL-11 Day 3 | 0.60 ± 0.55# | 1.00 ± 0.00# | 0.20 ± 0.45# | 0.20 ± 0.45* | 2.0 ± 1.34# |

*sig < Control Day 2.
#sig < Controls Day 2 & Day 3.
**sig < Control Day 2.
***Three vehicle-treated and five rhIL-11-treated animals were killed at each time point after receiving two doses rhIL-11 (37.5 μg/kg) or vehicle (2 days treatment). The histological analysis was performed without prior knowledge of the sample type and scored as follows: ulceration (0-2), inflammation (0-3), lesion depth (0-3), and fibrosis (0-2). A score of 0 denotes no lesion.

TABLE 4
rhIL-11-affected disease-related genes in the HLA-B27 rat*

| Gene | Symbol | Group | Fold Δ A | P value | Fold Δ B |
|---|---|---|---|---|---|
| Pancreatic | Pla2g1b | 11 | 202.67 | 0.0002 | 122.46 |
| phospholipase A2 | | | | | |
| Chymotrypsin B | Ctrb | 14 | 189.92 | 0.0002 | 136.17 |
| Pancreatic amylase | Amy1 | 13 | 185.92 | 4.3 E−6 | 64.85 |
| Syncollin | Sip9 | 10 | 104.92 | 7.3 E−6 | 75.25 |
| Elastase II | Ela2 | 14 | 96.76 | 3.6 E−5 | 51.77 |
| Pancreatic | Prss2 | 14 | 71.16 | 0.001 | 38.75 |
| trypsinogen II | | | | | |
| Pancreatic cationic | Try3 | 14 | 68.67 | 0.0003 | 64.56 |
| trypsinogen | | | | | |
| Carboxypeptidase A1 | Cpa1 | 14 | 67.65 | 0.0003 | 59.66 |
| Caldecerin | Ctrc | 14 | 62.50 | 0.0004 | 52.31 |
| Cholesterol | Cel | 11 | 54.06 | 0.0007 | 45.84 |
| esterase | | | | | |
| Pancreatic lipase | Pnlip | 11 | 48.76 | 9.7 E−6 | 82.59 |
| Elastase I | Ela1 | 14 | 46.34 | 0.0006 | 34.35 |
| Colipase | Clps | 11 | 42.04 | 0.0002 | 35.06 |
| Pancreatic | Prss1 | 14 | 35.93 | 8.3 E−6 | 29.16 |
| trypsin I | | | | | |
| Caboxypeptidase A2 | Cpa2 | 14 | 32.36 | 4.8 E−5 | 23.06 |
| Zymogen granule | Gp2 | 10 | 19.50 | 0.0002 | 33.38 |
| membrane protein (GP-2) | | | | | |
| Glycosylate membrane- | Pnliprp2 | 11 | 18.00 | 0.003 | 10.44 |
| associated lipase | | | | | |
| Pancreatic secretory | Spink2 | 15 | 12.66 | 0.001 | 2.55 |
| trypsin inhibitor type II | | | | | |
| Aquaporin 3 | Aqp3 | 22 | 3.77 | 0.010 | 4.61 |
| Plasmolipin | Z49858 | 21 | 3.67 | 0.046 | 7.89 |
| Isovaleryl-CoA | Ivd | 14 | 2.63 | 0.011 | −2.00 |
| dehydrogenase | | | | | |
| Cutaneous fatty acid- | Fabp-5 | 11 | −2.48 | 0.013 | −2.51 |
| binding protein (C-FABP) | | | | | |
| High mobility | Hmgiy | 5 | −2.53 | 0.022 | −1.77 |
| group protein I (Y) | | | | | |
| I-kappa B alpha chain | Nfkβla | 6 | −2.55 | 0.017 | −1.76 |
| Hexokinase type II | Hk2 | 13 | −2.71 | 0.024 | −3.20 |
| MHC class II-like | RT1.DMβ | 1 | −4.05 | 0.016 | −13.85 |
| beta chain (RT1.DMb) | | | | | |
| The names and symbols of 26 disease-related genes that rhIL-11-treatment specifically modulated are shown in columns one and two, respectively. The functional groups in which the genes are classified in Table 2 is shown, along with the fold-change modulation produced by rhIL-11-treatment of HLA-B27 rats (A) and the relative fold-change in the nondiseased Fischer 344 colon compared to the vehicle-treated HLA-B27 rat colon (B). Significance was | | | | | |
| # determined by a student t-test, and the resultant p values are shown. There was no significant difference between the rhIL-11-modulated levels of these genes in the HLA-B27 rat colon and their levels in the nondiseased colon of the Fischer 344 control. | | | | | |

TABLE 5
rhIL-11-affected nondisease-related genes in the HLA-B27 rat*

| | | | Fold Δ |
|---|---|---|---|
| Gene | Symbol | Accession No. | (relative to control) |
| Regeneration | RegIII | D23676 | 31.00 |
| protein III | | | |
| Insulin II | Ins2 | AI014020 | 2.52 |
| Glutathione | Gss | L38615 | −2.40 |
| synthetase | | | |
| VL30 element | VL30 | M91234 | −2.96 |
| ps20 | ps20 | AF037272 | −3.00 |
| Plakoglobin | Jup | U58858 | −3.33 |

*The name and symbol of each gene are shown with a fold-change value. RegIII and Ins2 were called “absent” in the vehicle-treated rat, therefore the fold-change values for these genes are much larger than presented.

What is claimed is:

1. A method of treating a subject suffering from IBD comprising administering RegIII protein or Ins2 protein to the subject.

2. A method of treating a subject suffering from IBD comprising administering to the subject a plurality of proteins selected from the group consisting of RegIII, Ins2, RegI, and TFF2, provided said plurality of proteins does not contain only RegI and TFF2.

3. The method of claim 2, wherein the plurality of proteins comprises a combination of proteins.

4. A method of treating a subject diagnosed with IBD, the method comprising administering to the subject an isolated nucleic acid molecule encoding RegIII or Ins2 operably linked to at least one expression control sequence, wherein the RegIII protein or the Ins2 protein is expressed in the subject.

5. A method of treating a subject diagnosed with IBD comprising administering to the subject a plurality of isolated nucleic acid molecules encoding proteins selected from the group consisting of RegIII, Ins2, RegI, and TFF2, operably linked to at least one expression control sequence, provided said plurality of isolated nucleic acid molecules encoding proteins does not contain only RegI and TFF2.

6. A method of identifying a compound capable of increasing the activity of a protein selected from the group consisting of RegIII and Ins2 comprising the steps of:
(a) contacting a sample containing the protein with one of a plurality of test compounds; and
(b) comparing the activity of the protein in the contacted sample with that in a sample containing the protein not contacted with the test compound,
wherein a substantial increase in the activity of the protein in the contacted sample identifies the compound as an activator of protein activity useful in treating IBD.

7. A method of treating a subject suffering from IBD comprising administering to the subject a compound identified by the method of claim 6.

8. A method of identifying a compound capable of increasing the activities of a plurality of proteins selected from the group consisting of RegIII, Ins2, RegI, and TFF2, provided said plurality of proteins does not contain only RegI and TFF2, comprising the steps of:
(a) contacting a sample containing the plurality of proteins with one of a plurality of test compounds; and
(b) comparing the activities of the plurality of proteins in the contacted sample with those in a sample containing the plurality of proteins not contacted with the test compound,
wherein increases in the activities of the plurality of proteins in the contacted sample identify the compound as an activator of protein activity useful in treating IBD.

9. A method of treating a subject suffering from IBD comprising administering to the subject a compound identified by the method of claim 8.

10. A method of treating a subject suffering from IBD comprising administering to the subject a compound that increases the activity of RegIII protein and/or the activity of Ins2 protein.

11. A method of treating a subject diagnosed with IBD, the method comprising administering a composition comprising a compound that modulates the activity of an IBD marker polypeptide and a pharmaceutically acceptable carrier, wherein the IBD marker is listed in Table 4 and expression of the IBD marker was modulated by rhIL-11 treatment in Table 4.

12. The method of claim 11, wherein the IBD marker is selected from the group consisting of HLA-DMβ and RT1.DMβ.

13. A method of treating a subject diagnosed with IBD, the method comprising administering a composition comprising an IBD marker polypeptide and a pharmaceutically acceptable carrier, wherein the IBD marker polypeptide is encoded by an IBD marker listed in Table 4 and expression of the IBD marker was increased by rhIL-11 treatment in Table 4.

14. A method of treating a subject diagnosed with IBD, the method comprising administering to the subject an isolated nucleic acid molecule encoding an IBD marker listed in Table 4 operably linked to at least one expression control sequence, wherein the IBD marker protein is expressed in the subject, and wherein expression of the IBD marker was increased by rhIL-11 treatment in Table 4.

15. A composition capable of inhibiting IBD in a subject, the composition comprising an IBD marker polypeptide and a pharmaceutically acceptable carrier, wherein the IBD marker polypeptide is encoded by an IBD marker listed in Table 4 and expression of the IBD marker was increased by rhIL-11 treatment in Table 4.

16. A composition capable of inhibiting IBD in a subject, the composition comprising a siRNA molecule and a pharmaceutically acceptable carrier, wherein the siRNA molecule is targeted to a mRNA corresponding to an IBD marker listed in Table 4 and expression of the IBD marker was decreased by rhIL-11 treatment in Table 4.

17. The method of claim 16, wherein the IBD marker is selected from the group consisting of HLA-DMβ and RT1.DMβ.

18. A method of treating a subject diagnosed with IBD, the method comprising providing to the subject a polynucleotide that inhibits expression of an IBD marker, wherein the IBD marker is listed in Table 4 and expression of the IBD marker was decreased by rhIL-11 treatment in Table 4.

19. A method of treating a subject diagnosed with IBD, the method comprising providing to the subject a siRNA molecule that inhibits expression of an IBD marker, wherein the siRNA molecule is targeted to a mRNA corresponding to an IBD marker listed in Table 4 and expression of the IBD marker was decreased by rhIL-11 treatment in Table 4.

20. A method of treating a subject diagnosed with IBD, the method comprising providing to the subject an antisense oligonucleotide complementary to an IBD marker, wherein the IBD marker is listed in Table 4 and expression of the IBD marker was decreased by rhIL-11 treatment in Table 4.

21. A method of treating a subject diagnosed with IBD, the method comprising providing to the subject an antibody capable of immunospecific binding to an IBD marker protein, wherein the IBD marker protein is encoded by an IBD marker listed in Table 4 and expression of the IBD marker was decreased by rhIL-11 treatment in Table 4.

22. A method of identifying a compound capable of increasing expression of an IBD marker selected from the group consisting of RegIII and Ins2 comprising the steps of:
(a) contacting a sample containing the IBD marker with one of a plurality of test compounds; and
(b) comparing the level of expression of the IBD marker in the contacted sample with that in a sample containing the IBD marker not contacted with the test compound,
wherein a substantial increase in the level of expression of the IBD marker in the contacted sample identifies the compound as useful in treating IBD.

23. A method of treating a subject diagnosed with IBD comprising administering to the subject a compound identified by the method of claim 22.

24. A method of identifying a test compound for inhibiting IBD comprising the step of comparing:
a) a level of expression of an IBD marker, wherein the IBD marker is listed in Tables 4 or 5, in a first sample, wherein the first sample is contacted with one of a plurality of test compound; and
b) a level of expression of the same IBD marker in a second sample, wherein the second sample is not contacted with the test compound,
wherein a substantially modulated level of expression of the IBD marker in the first sample, relative to the second sample, is an indication that the test compound is efficacious for inhibiting IBD.

25. The method of claim 24, wherein the IBD marker is selected from the group consisting of HLA-DMβ and RT1.DMβ, and wherein the substantially modulated level of expression is a substantially decreased level of expression.

26. The method of claim 24, wherein the IBD marker is selected from the group consisting of RegIII and Ins2, and wherein the substantially modulated level of expression is a substantially increased level of expression.
